(19) United States
(12) Patent Application Publication
(10) Pub. No.: US 2013/0310691 A1
(43) Pub. Date: Nov. 21, 2013

(54) SYSTEM, METHOD AND DEVICE FOR AUTOMATIC AND AUTONOMOUS DETERMINATION OF HEMODYNAMIC AND CARDIAC PARAMETERS USING ULTRASOUND

(71) Applicant: Cardio Art Technologies Ltd., Or Yehuda (IL)

(72) Inventors: Dan Gur Furman, Gedera (IL); Nissim Varon, Kfer Saba (IL); Lev Adirovich, Gan Yavne (IL); Alex Roytvarf, Rishon Lezion (IL)

(21) Appl. No.: 13/951,757

(22) Filed: Jul. 26, 2013

Related U.S. Application Data
(63) Continuation of application No. PCT/IB2012/000124, filed on Jan. 27, 2012.
(60) Provisional application No. 61/437,318, filed on Jan. 28, 2011.
(60) Provisional application No. 61/480,713, filed on Apr. 29, 2011.

(51) Int. Cl. A61B 8/08; A61B 8/00; A61B 8/06

(52) U.S. Cl. CPC A61B 8/0891; A61B 8/06; A61B 8/4477; A61B 8/488
USPC 600/447; 600/449

(57) ABSTRACT

The present disclosure relates to an ultrasound device, system and a method for determination of cardiac and/or hemodynamic parameters, and in particular, to such a system, and method in which the cardiac and/or hemodynamic parameters are determined in a non-invasive manner that is both automatic and autonomous and, therefore, does not depend on ultrasound imagery and/or a skilled caregiver analysis thereof.

### CROSS-REFERENCE TO RELATED APPLICATIONS

This application is a continuation of International Application PCT/IB12/00124, with an international filing date of Jan. 27, 2012, which claims the benefit of priority from U.S. Provisional Patent Application Ser. No. 61/437,318 filed Jan. 28, 2011 and U.S. Provisional Patent Application Ser. No. 61/480,713 filed Apr. 28, 2011; the disclosures of said applications are expressly incorporated by reference herein in their entirety.

### BACKGROUND AND SUMMARY

The present disclosure relates to an ultrasound system, device and a method for determining cardiac and/or hemodynamic parameters, and in particular, to such a system, device and method in which the cardiac and/or hemodynamic parameters are determined in a non-invasive or minimally invasive manner.

Cardiac monitoring is required for obtaining essential cardiac and hemodynamic parameters in monitoring and/or treating a patient. Such cardiac monitoring may be provided by different techniques of varying levels of invasiveness for example invasive, minimally invasive and non-invasive. For example, angiography, angiogram, cardiac catheterization, right heart catheterization, left heart catheterization may be considered invasive or minimally invasive cardiac procedures. Alternatively, ultrasound based techniques such as echocardiogram, may be considered non-invasive method for determining available cardiac and/or hemodynamic parameters.

Individual cardiac monitoring techniques have their advantages and disadvantages. Particularly the invasive and/or minimally invasive techniques may provide accurate cardiac and/or hemodynamic parameters, however, the level of invasiveness may greatly deter individuals and practitioners from undertaking such procedures unless absolutely necessary and where no alternative is available.

One alternative to invasive measures and techniques includes the use of ultrasongraphy and, in particular, Doppler ultrasound in non-invasive techniques, such as echocardiogram. While such ultrasound technology offers and improves upon the invasive nature of cardiac monitoring via catheterization, it is limited in that the accuracy of the monitored cardiac and/or hemodynamic parameters greatly depends on the skill, experience and expertise of the practitioner and/or technician performing the procedure. Therefore, the reliability and repeatability of the cardiac and/or hemodynamic parameters are greatly influenced and limited by the skill level and acumen of the practitioner performing the scan. Moreover, due to the limitation imposed by a practitioner's skill level, current echocardiograms cannot and do not provide the same cardiac and/or hemodynamic parameters as that offered by the invasive methods, therefore maintaining the need for the invasive and minimally invasive procedures. Accordingly, some cardiac and/or hemodynamic parameters are not available by non-invasive means. Such parameters, for example, intra-cardiac pressure, may only be measured in an invasive manner, where the Swan-Ganz Catheter provides the gold standard procedure.

The system does not require the involvement of a skilled healthcare giver to determine cardiac and/or hemodynamic parameters, rather the system, device and method according to a preferred embodiment of the present disclosure provide for automatic, autonomous determination of cardiac and/or hemodynamic parameters.

Within the context of this application, the term ‘static scanning area’ or ‘stationary scanning area’ may be used interchangeably to refer to an area about a subject's torso that is being scanned with an ultrasound probe, where the ultrasound probe is essentially maintained in a static and/or stationary position while performing the scan. The ultrasound scan may be performed by a user, physician, caregiver, trained technician, skill artisan, or performed by the subject himself, wherein the probe is essentially stationary and/or static in one location about the torso. Therefore, the scan is performed essentially without movement of a probe by the person performing the ultrasound scan, therein essentially providing for an ultrasound scan over a stationary or otherwise static location, for example, along subject's torso, chest, back side or the like. The scanned area is a static scanned area over a subject's thoracic region, for measuring, obtaining and/or determining hemodynamic and/or cardiac parameters. Optionally, the system according to the present disclosure may be utilized to measure, obtain and/or determine a plurality of parameters about any scanning area about a subject.

Within the context of this application, the term ‘low resolution mask detection’ refers to the process of assessing ultrasound reflected data signals with a low resolution mask and/or filter, for example, a low resolution edge detection filter.

Within the context of this application, the term ‘high resolution mask detection’ refers to the process of assessing ultrasound reflected data signals with a high resolution mask and/or filter, for example, a high resolution edge detection filter.

Within the context of this application, the term ‘about’ when referring to a value, refers to plus or minus 10% of the value cited

Within the context of this application, the term ‘autonomous’ refers to an intrinsic method, process or calculation carried out or performed independently of and without the assistance of a user.

Within the context of this application, the term ‘ultrasound transducer’ may refer to any type of ultrasound transducer as is known and accepted in the art, for example, including, but not limited, to one dimensional and/or two dimensional and/or three dimensional transducers comprising a plurality of piezoelectric elements and/or crystals as is known and accepted in the art.

Within the context of this application, the term ‘ultrasound producing elements’ may, for example, include, but are not limited to, Capacitive Micro-machined Ultrasonic Transducer (‘cMUT’), piezoelectric crystals, ceramics, or the like.

Within the context of this application the term ‘Rvir’ or virtual Radius herein refers to a vessel radius under absolute conditions where the pressure is equal to about zero.

Within the context of this application the term ‘Rdia’ is interchangeable with diastolic Radius herein refers to vessel radius during the cardiac cycle diastole.

Within the context of this application the term ‘Rsys’ is interchangeable with systolic Radius herein refers to a vessel radius during the cardiac cycle systole.

Within the context of this application the term ‘Tpp’, ‘tpp, or pulse pressure time herein refers to the length of time during cardiac cycle pulse pressure.

Within the context of this application the term ‘Tsys’, ‘tsys’, or systole time herein refers to the length of time during cardiac cycle systole.

Within the context of this application the terms ‘R_{P7}’ and/or, ‘P7’ herein refers to vessel radius corresponding to the an ideal blood vessel radius at minimal pressure.

Within the context of this application the following shorthand references are used to refer to the defined term as commonly understood, known and accepted in the art as detailed below: P=Pressure; PP—pulse pressure; Psys—systolic pressure; Pdia—diastolic pressure; δ—vessel deformation change; ρ—blood density; c—Pressure wave propagation velocity; En—effective Yang modules; Rvir—virtual vessel radius; Rsys/Rs—systolic radius; Rdia/Rd—diastolic radius; h—vessel wall thickness; Us—systolic blood flow velocity; Ud—diastolic blood flow velocity; λ—Yang module coefficient; k—constant.

Although the foregoing description provides specific examples for the measuring, obtaining and/or determination of cardiac and/or hemodynamic parameters based on ultrasound scanning of a subject's thoracic region, the system and method of the present disclosure is not limited to such application.

An optional embodiment of the present disclosure provides for a non-invasive ultrasound system for automatic and autonomous determination of cardiac and/or hemodynamic parameters with or without presenting an image of a scanned area to a user and wherein the scanned area is a static area over a subject's thoracic region, for example, the chest, the system comprising: an ultrasound probe including a plurality of ultrasound transducers for scanning the static area over the chest; and a probe scan engine for controlling the plurality of ultrasound transducers and for processing data obtained from the ultrasound transducers to produce a set of vessel parameters; and a processor for inferring and/or determining cardiac and/or hemodynamic parameters from the vessel parameters.

An optional embodiment of the present disclosure provides for a non-invasive ultrasound probe for automatic and autonomous determination of cardiac parameters without presenting an image of a scanned area to a user and wherein the scanned area is a static area over the chest, the probe including a single housing comprising a plurality of ultrasound transducers for scanning the static area over the chest.

An optional embodiment of the present disclosure provides for a non-invasive method for automatically and autonomously determining cardiac and/or hemodynamic parameters of a subject based on a combination of ultrasound and Doppler ultrasound scan of a static scanning area over the upper torso, for example, the chest of a subject, wherein the scan is performed without presenting an ultrasound image of the static scanning area to a user and/or practitioner and/or caregiver, the method comprising: scanning a static scanning area over the chest of a subject with an ultrasound probe comprising an array of a plurality of ultrasound transducers and autonomously determining at least two vessel parameters for at least one vessel within the static scanning area; and further processing the at least two vessel parameters for at least one vessel to determine and/or elucidate cardiac and/or hemodynamic parameters of the subject.

Unless otherwise defined, the various embodiment of the present disclosure may be provided to an end user in a plurality of formats, platforms, and may be outputted to at least one of a computer readable memory, a computer display, a printout, a computer on a network or a user.

The materials, methods, and examples provided herein are illustrative only and not intended to be limiting.

Implementation of the method and system of the present disclosure involves performing or completing certain selected tasks or steps manually, automatically, or a combination thereof. Moreover, according to actual instrumentation and equipment of preferred embodiments of the method and system of the present disclosure, several selected steps could be implemented by hardware or by software on any operating system of any firmware or a combination thereof. For example, as hardware, selected steps of the disclosure could be implemented as a chip or a circuit. As software, selected steps of the disclosure could be implemented as a plurality of software instructions being executed by a computer using any suitable operating system. In any case, selected steps of the method and system of the disclosure could be described as being performed by a data processor, such as a computing platform for executing a plurality of instructions.

### BRIEF DESCRIPTION OF THE DRAWINGS

The embodiments of the present disclosure are herein described, by way of example only, with reference to the accompanying drawings. With specific reference now to the drawings in detail, it is stressed that the particulars shown are by way of example and for purposes of illustrative discussion of the preferred embodiments of the present disclosure only, and are presented in order to provide what is believed to be the most useful and readily understood description of the principles and conceptual aspects of the embodiments. The description taken with the drawings make it apparent to those skilled in the art how the several forms of the embodiments of the present disclosure may be embodied in practice.

In the drawings:

FIG. 1A-1C are schematic block diagrams of an exemplary system for automatic and autonomous determination of hemodynamic and cardiac parameters using ultrasound;

FIG. 2A-2D are schematic block diagrams of an exemplary probe of the system;

FIG. 2E-2F are schematic block diagrams of an alternative embodiment of the exemplary probe;

FIG. 3 is an exemplary method for automatically and autonomously determining a subject's cardiac and/or hemodynamic parameters;

FIG. 4A-4B are flowcharts of an exemplary methods for automatic scanning of a static area over a subject's chest;

FIG. 4C is a schematic illustrative block diagram depicting the method of FIG. 4A;

FIG. 5A is a flowchart of an exemplary method for automatic scanning a static area over a subject's chest for determining vessel diameter and center;

FIG. 5B is a schematic illustrative block diagram depicting the method of FIG. 5A;

FIG. 6A is a flowchart of an exemplary method for automatically scanning a static area over a subject's chest for determining vessel parameters;

FIG. 6B is a schematic illustrative block diagram depicting the method of FIG. 6A;

FIG. 7 is a flowchart of an exemplary method for determining hemodynamic and cardiac parameters automatically based on Doppler ultrasound;

FIG. 8 is a schematic illustrative diagram of a graph of a velocity time curve utilized for automatically determining hemodynamic and cardiac parameters as depicted in FIG. 7;

FIG. 9. is a flowchart of an exemplary method for determining and calculation hemodynamic and cardiac parameters automatically based on Doppler ultrasound;

FIG. 10 is a flowchart of an exemplary method for automatic scanning a static area over a subject's chest for identifying at least one or more vessel objects;

FIG. 11 is a flowchart of an exemplary method for automatic scanning a static area over a subject's chest for targeting and following a vessel of interest over time;

FIG. 12A-B are schematic illustration of the method for targeting and following a vessel of interested as described in FIG. 11;

FIG. 13 is a flowchart of an optional method for processing vessel objects data for automatically determining hemodynamic and cardiac parameters based on ultrasound and Doppler scans according to an optional embodiment of the present disclosure;

FIG. 14 shows an illustrative schematic diagram according to optional embodiments of the present disclosure; and

FIG. 15 is a flowchart of an optional method for processing vessel objects data for automatically determining hemodynamic and cardiac parameters based on ultrasound and Doppler scans according to an optional embodiment of the present disclosure.

### DETAILED DESCRIPTION OF EMBODIMENTS OF THE DISCLOSURE

The principles and operation of the present disclosure may be better understood with reference to the drawings and the accompanying description. The following reference labels listed below are used throughout the drawings to refer to objects having similar function, meaning, role, or objective.
n1-n8 Ultrasound transducers;
100 Automatic and Autonomous ultrasound system;
102 System Management Processor;
104 User Interface;
110 Ultrasound probe;
112 Scan engine;
114 US transducer array;
115 IR sensor array;
116 Multiplexer;
118 Probe controller;
120 Decision Support System;
800 Velocity time curve;
802a-b Cardiac cycle curve segments;
802a Pulse Pressure segment;
802b Pressure Drop segment;
804a-d Extremum point, min and max points;
804a, d Diastole point;
804b Maximum velocity;
804c Valve closure;
806 Cardiac cycle sub-segments (i);
n Plurality of ultrasound transducers;
n−x Subset of a plurality of ultrasound transducers;
sl Scan lines;
dsl Doppler scan lines;
fdsl Flanking Doppler scan lines;
cl Chord length based scan line;
v Vessel of interest;
vc Vessel center;
vr Vessel radius;
z An optimal transducer of n transducers.

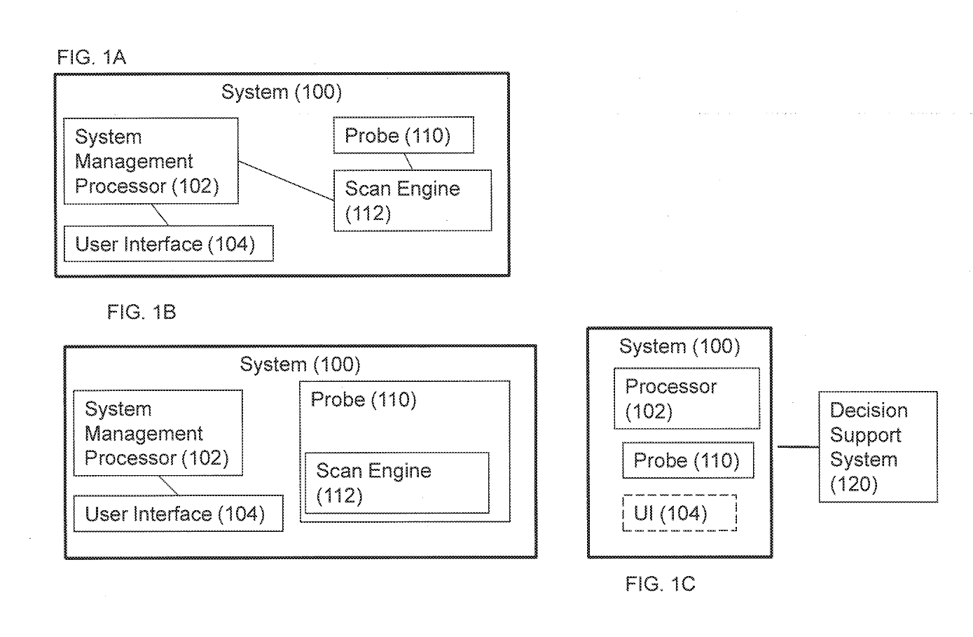

Referring now to the drawings, FIG. 1A shows a schematic block diagram of a system 100, according to one embodiment of the present disclosure, comprising an ultrasound scanning probe 110 customized and/or specific to system 100, and a system management processor 102. System 100 provides for scanning an area on a subject with ultrasound probe 110 in order to obtain data about the scanned area. The scanned area is a static scanned area over a subject's thoracic region, for measuring, obtaining and/or determining hemodynamic and/or cardiac parameters. Optionally, the system may be utilized to measure, obtain and/or determine a plurality of parameters about any scanning area about a subject.

Although the foregoing description provides specific examples for the measuring, obtaining and/or determination of cardiac and/or hemodynamic parameters based on ultrasound scanning of a subject's thoracic region, the system and method of the present disclosure is not limited to such application. System 100 may be utilized to scan any other region about the subject's body to determine a plurality of parameters associated with the scanned area within the scanning region, for example, including, but not limited to, extremities.

System management processor 102 is provided with and/or otherwise coupled with a user interface 104. System management processor 102 may, for example, include, but is not limited to, a computer, personal digital assistant (PDA), mobile computer, mobile processing device, mobile communication device, mobile telephone or the like device comprising a processor for processing data, managing data flow and/or controlling flow and processing of information from a plurality of sources.

Optionally, at least one or more user interface 104 may be provided in at least one of optional forms, for example, including, but not limited to, a display, keyboard, mouse, speakers or the like devices known in the art providing for human interface with system 100 and, in particular, with system management processor 102.

Probe 110 is provided in the form of an ultrasound probe that is controllable with a scan engine 112. Optionally scan engine 112 communicates with system management processor 102. Optionally communication between scan engine 112 and system management processor 102 may be provided in at least any one or more forms of wired, wireless, cellular, or the like communication methods and/or protocols.

Probe 110 comprises a plurality of ultrasound transducers. Optionally, probe 110 comprises at least one ultrasound transducer. Probe 110 may comprises at least 4 or more ultrasound transducers. Optionally, probe 110 may comprise up to 10 ultrasound transducers. Illustratively, probe 110 comprises 8 ultrasound transducers, n1, n2, n3, n4, n5, n6, n7, and n8. Probe 110 comprises at least two or more ultrasound transducers, for example 2, or 3, or 4, or 5, or 6, or 7, or 8 or, 9, or 10 individual ultrasound transducers.

Optionally, each ultrasound transducer is provided with a plurality of ultrasound elements in the form of piezoelectric elements and/or crystals or the like ultrasound generating material, as is known and accepted in the art. Optionally, each ultrasound transducer provided with probe 110 may, for example, comprise at least 32, and up to about 256, ultrasound elements, and more particularly, 48 or more ultrasound elements. For example, ultrasound elements may be a piezoelectric element, cMUT or the like ultrasound generating device. The ultrasound elements and transducers collectively provide for producing ultrasound scan-lines (sl) over a given scanned area. Optionally probe 110 may comprise at least one or more of one dimensional (1D) ultrasound transducers, two dimensional (2D) ultrasound transducers, and/or three dimensional (3D) ultrasound transducers, or any combination thereof.

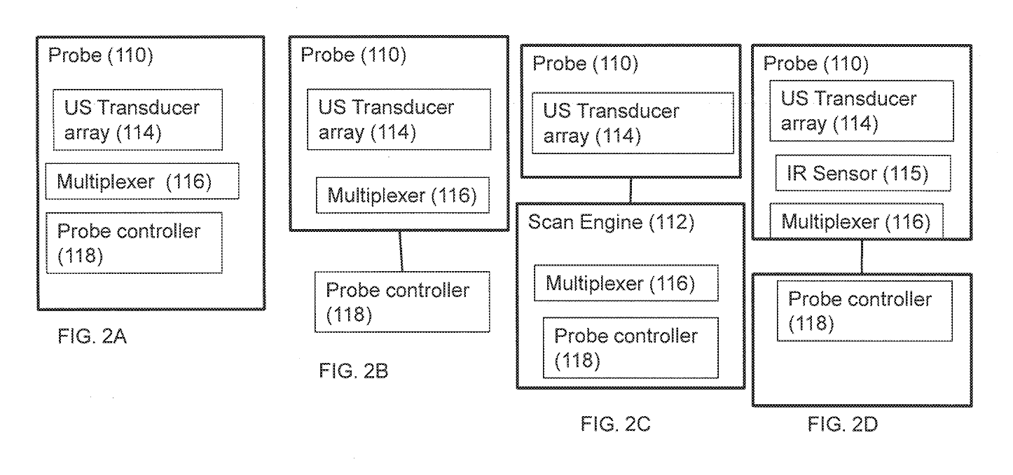

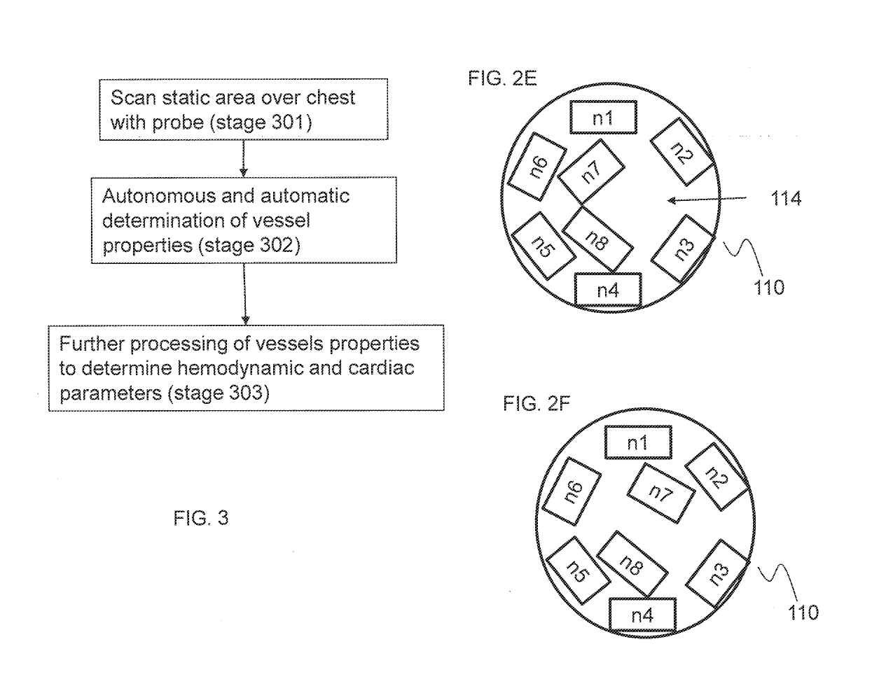

Individual ultrasound scan-lines may be selectively and specifically controlled with scan engine 112. Scan engine 112 provides for controlling probe 110 by controlling a plurality of ultrasound transducers and ultrasound elements. A multiplexer 116 (FIG. 2A-2D) is associated and/or otherwise interfaces, or coupled with the scan engine 112 and probe 110 to control the individual ultrasound elements and the scan-lines produced therefrom.

Probe 110 may, for example, produce a sector scan phased array ultrasound and/or Doppler ultrasound signals, or the like signal.

Probe 110 provides for a non-invasive tool to provide data and/or information that may be utilized in evaluating and/or monitoring a scanning area of a subject, for example, a human and/or animal. The scanning area is determined by the placement of probe 110 over the subject, in order to allow the ultrasound elements to generate and/or detect reflected ultrasound signal within and/or about the scanning area. Probe 110 may be utilized to evaluate and/or monitor the ultrasound signal and/or data provided by and obtained from the scanned area.

Optionally, the ultrasound signal and/or data may provide for abstracting an ultrasound image and/or a Doppler ultrasound image of the scanned area. Optionally, an abstracted ultrasound image may be displayed to a system operator, for example, a healthcare provider, doctor, nurse, technician with user interface 104 in the form of an image.

The ultrasound signal and/or data provides for abstracting ultrasound and/or Doppler data that are not associated with an image and/or a Doppler ultrasound image of the scanned area. Optionally, abstracted ultrasound data may be displayed to a system operator, for example, with user interface 104 in the form of a display.

An optional embodiment of the present disclosure is shown in FIG. 1B where scan engine 112 is coupled or otherwise integrated with probe 110.

An optional embodiment of the present disclosure is shown in FIG. 1C where system 100 is further coupled to or otherwise associated with a decision support System 120. Optionally, decision support system 120 may be provided in the form of a back office, call center, health care provider, ambulatory care, telemedicine center, a medical decision support system, a caregiver decision support system, for example, to facilitate further processing, analysis and/or treatment decision support based on the parameters and data provided with system 100 as previously described.

In an alternative embodiment, system 100 provides for scanning a static or constant scan area over a scanning area, for example, over a subject's thoracic region. System 100 is adept for scanning a static scanning area without displaying an image, for example, an ultrasound image, to a system operator and/or subject. System 100 is adapted to provide for autonomous and automatic ultrasound and Doppler scanning of a static scanning area of a subject.

Additionally, system 100 provides for determining hemodynamic and/or cardiac parameters of a subject from a static scanning area over the subject's thoracic region, for example, the chest. An alternative embodiment provides for determining the hemodynamic and/or cardiac parameters from a static scanning area over the chest without displaying to a system operator any ultrasound signal, however, optionally, an image of the static scanning area may be provided to a subject and/or system operator.

FIGS. 2A-2D provide schematic depictions of optional configurations for probe 110 according to an optional embodiment of the present disclosure comprising at least two or more ultrasound transducers in an ultrasound transducer array 114 for generating and reading ultrasound signals, a multiplexer 116 for controlling the array of ultrasound transducers 114 and a probe controller 118 mediating control and processing of probe 110 with scan engine 112.

FIGS. 2A-2B show an optional configuration of probe 110 wherein the array of transducers 114 and multiplexer 116 are provided in a single housing while probe controller 118 may optionally be disposed in the same housing, as shown in FIG. 2A, or in a separate housing as shown in FIG. 2B, for example, as part of scan engine 112.

FIG. 2C provides an additional depiction of probe 110 as comprising the array of ultrasound transducers 114 in a single housing while multiplexer 116 and probe controller 118 are provided in a separate housing, for example, disposed as part of the scan engine 112.

FIG. 2D provides an additional optional embodiment, where probe 110 further comprises at least one or more IR sensor 115. Optionally, IR sensor 115 provides for determining blood saturation in order to facilitate identifying at least one or more vessels of interest within the probe scanning area.

FIGS. 2E-2F provide schematic depictions of probe 110 according to an optional embodiment of the present disclosure wherein the ultrasound probe comprises a plurality of ultrasound transducers (n), optionally at least two or more transducers. In particular, there may be four, six, eight, or ten ultrasound transducers. Illustratively, there are eight ultrasound transducers.

Although the description herewith provides a description of a probe that comprises 8 transducers, it is to be understood that the number of transducers utilized is for illustrative purpose such that the probe of the present disclosure is not limited to a predefined finite number of transducers. While the described functionality may be described with a probe comprising eight ultrasound transducers it may equally be realized with a fewer number of transducers, therefore, the embodiment of the present disclosure is not limited to 8 or 4 transducers and rather may be generalized to have a plurality of transducers comprising at least two or more ultrasound transducers.

An optional embodiment of the present disclosure provides for an ultrasound probe 110 comprising at least 2 transducers. Optionally, the transducers may be arranged relative to one another in a unique manner according to an optional embodiment of the present disclosure. Ultrasound probe 110 comprising at least 2 or more ultrasound transducers, may be arranged such that 6 transducers are disposed in a hexagonal configuration, such that each of the 6 transducers is disposed about a vertex of a hexagon and at least 2 or more transducers may be disposed along any two chords defined between the six hexagonal vertices.

Optionally, probe 110 may comprise at least one or more of one dimensional (1D) ultrasound transducers, two dimensional (2D) ultrasound transducers, and/or three dimensional (3D) ultrasound transducers, or any combination thereof.

Optionally, probe 110 may comprise a combination of 2D and/or 3D transducers individually arranged such that a plurality of two dimensional (2D) transducers may form a first arrangement while a plurality of three dimensional (3D) transducers may produce a second arrangement about probe 110. For example, probe 110 may comprise 6 transducers arranged in a six pointed star formation including a first triangular arrangement including three (3) 2D transducers and a second triangular arrangement including three (3) 3D transducers.

Optionally, probe 110 may produces at least 128 or more scan-lines controllable with scan engine 120.

An optional embodiment of probe 110 wherein each transducer (n) may be provided with a plurality of ultrasound producing elements, for example, including, but not limited to, piezoelectric crystals, piezoelectric ceramic, cMUT, or the like. For example, each transducer may comprise from about 32 to about 256 ultrasound piezoelectric elements.

Optionally, each of ultrasound transducer (n) utilized with probe 110 may comprise the same or a different number of piezoelectric elements. For example, an optional probe 110 comprising n ultrasound transducers where n=8 (n1-n8) may all have about 48 ultrasound elements. For example, an optional probe 110 comprising n ultrasound transducers where n=8 {n1 . . . n8} may be configured such that {n1, n3, n4, n7} may be provided with 64 ultrasound elements, while {n2,n5,n6} may be provided with 32 ultrasound elements, and {n8} may be provided with 128 ultrasound elements, or the like arrangement.

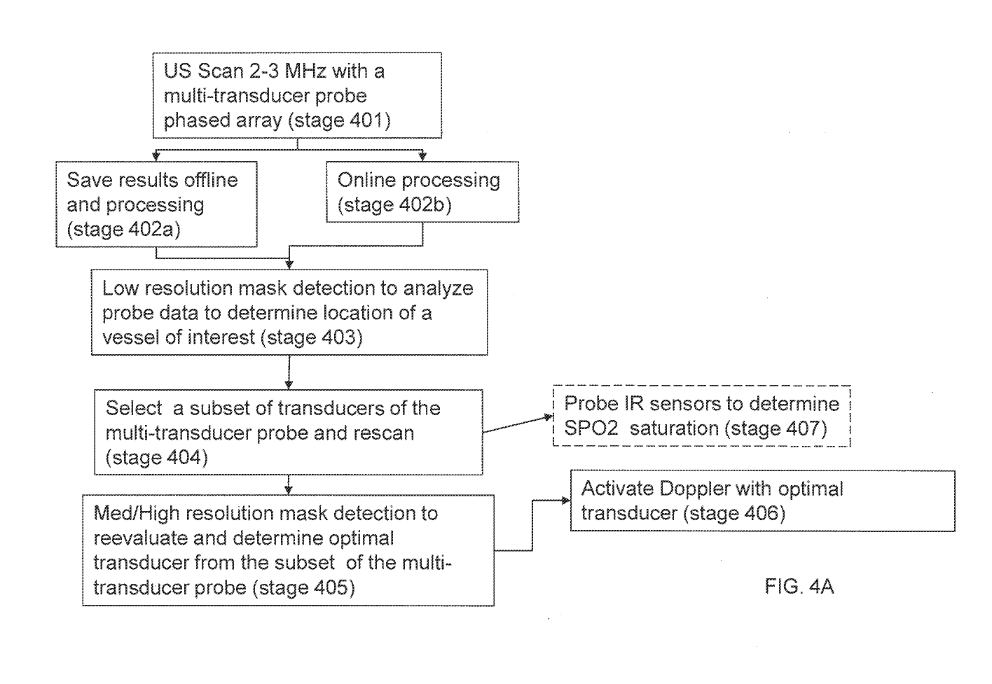

FIG. 3 depicts a method for determining hemodynamic and/or cardiac parameters with system 100 according to optional embodiments of the present disclosure, as shown in FIG. 1A-1C. Optionally, the method for measuring, obtaining and/or determination of cardiac and/or hemodynamic parameters based on ultrasound scanning of a subject's thoracic region with system 100 starts in stage 301 with a combination of an ultrasound and Doppler ultrasound scan of a static scanning area about a subject's thoracic region, utilizing probe 110 to perform the scan.

Optionally, the static scanning area is within any region and/or portion of the thoracic region, for example, including, but not limited to, the front, back, right side, left side, thoracic spine, chest, armpit or the like portion of the thoracic region. In particular, the static scanning area is performed over the chest.

In an alternative embodiment, the static scanning area is such that once a scanning area location is determined the probe is placed over the area and the scan is preformed over the scan area substantially without gross movement of the probe such that the probe is maintained in an essentially constant or static position relative to the surface of scanning region, for example, the thoracic region.

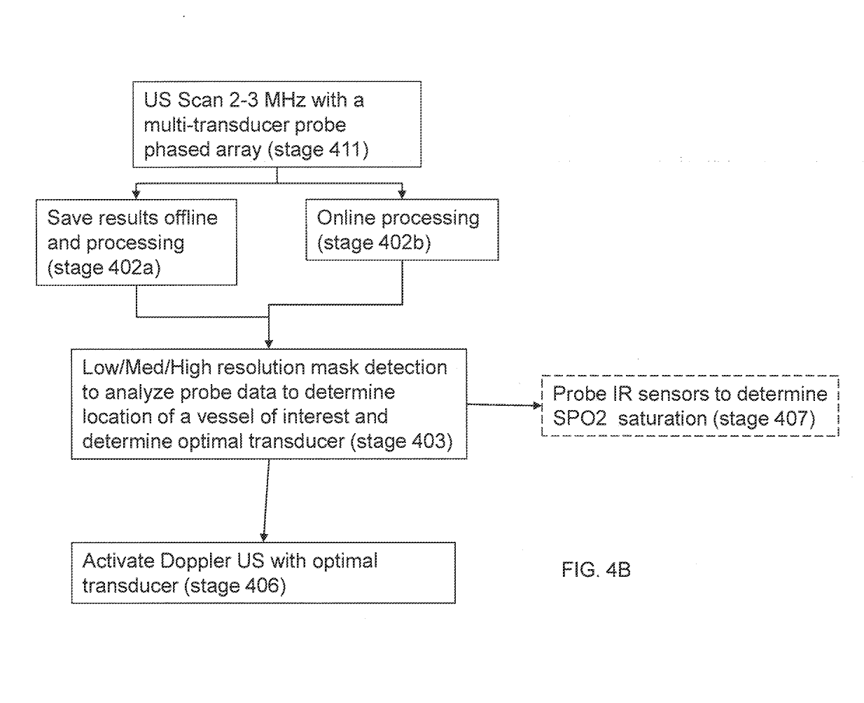

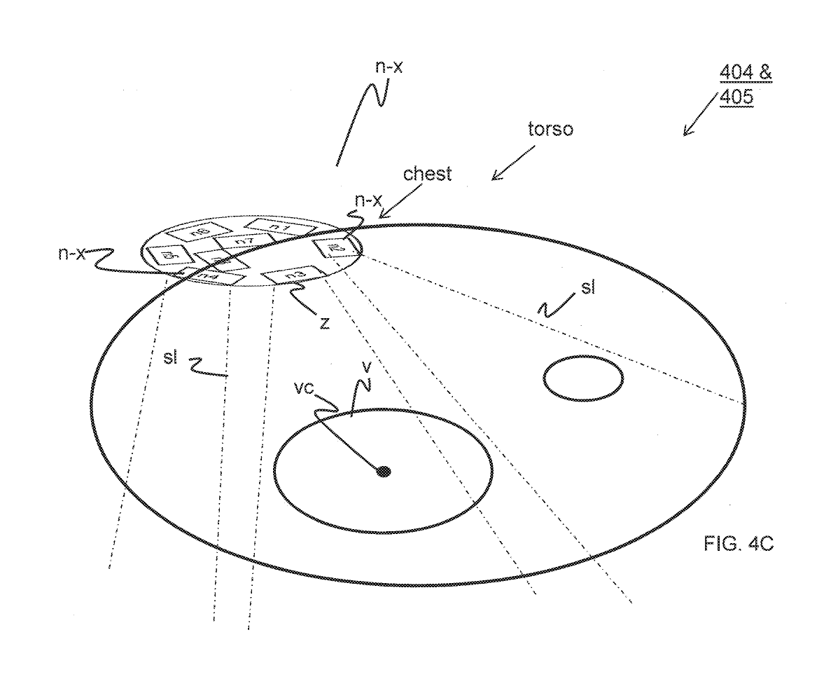

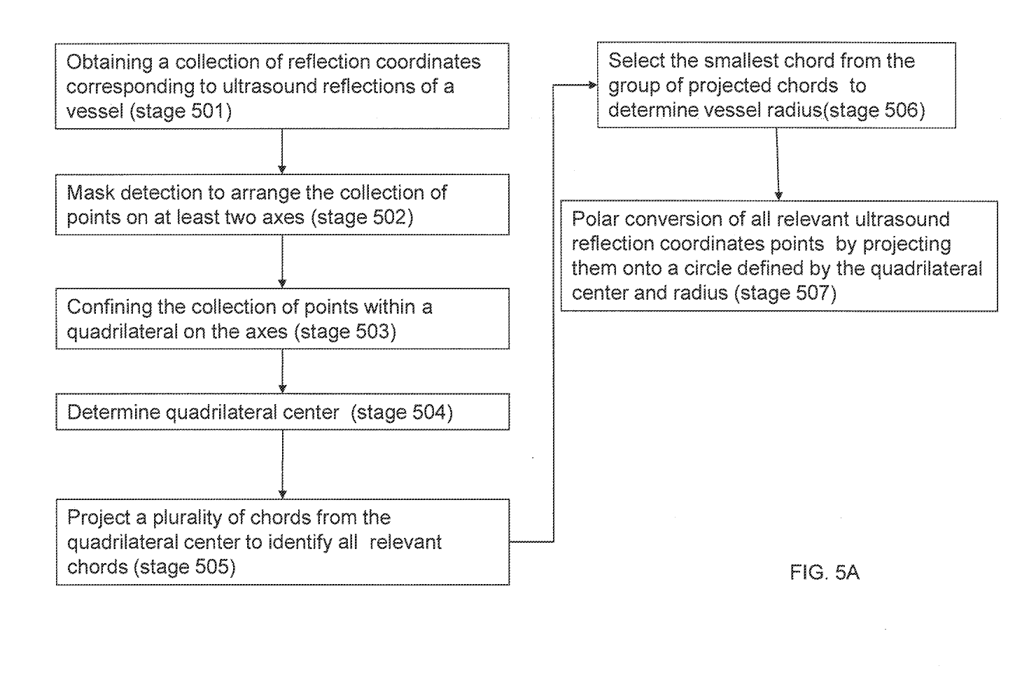

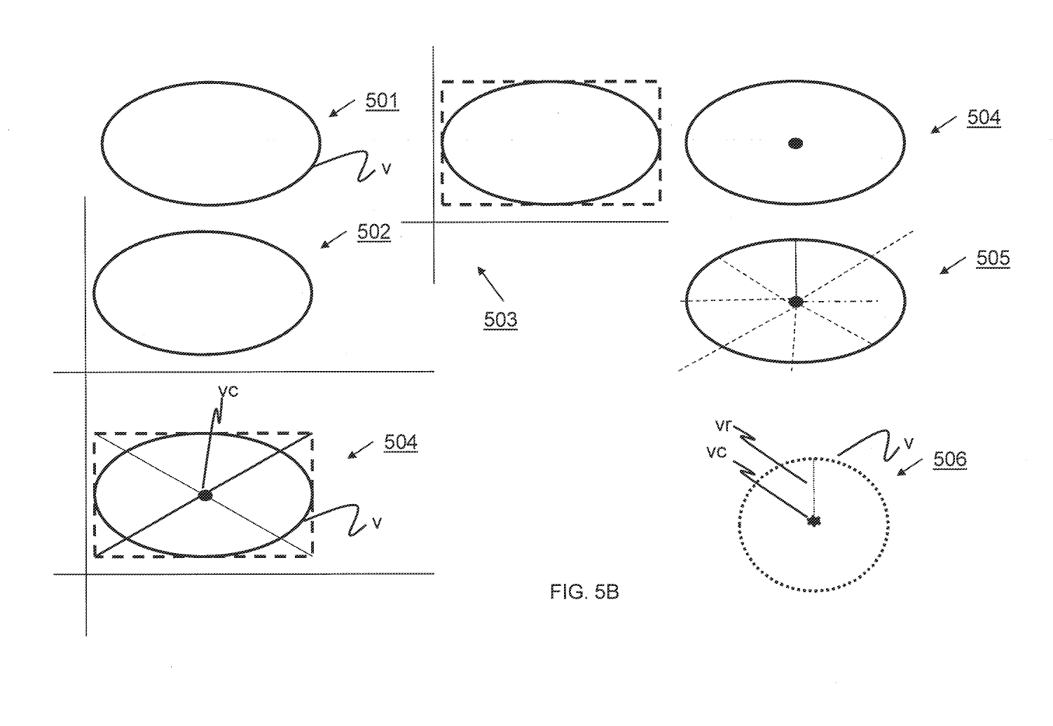

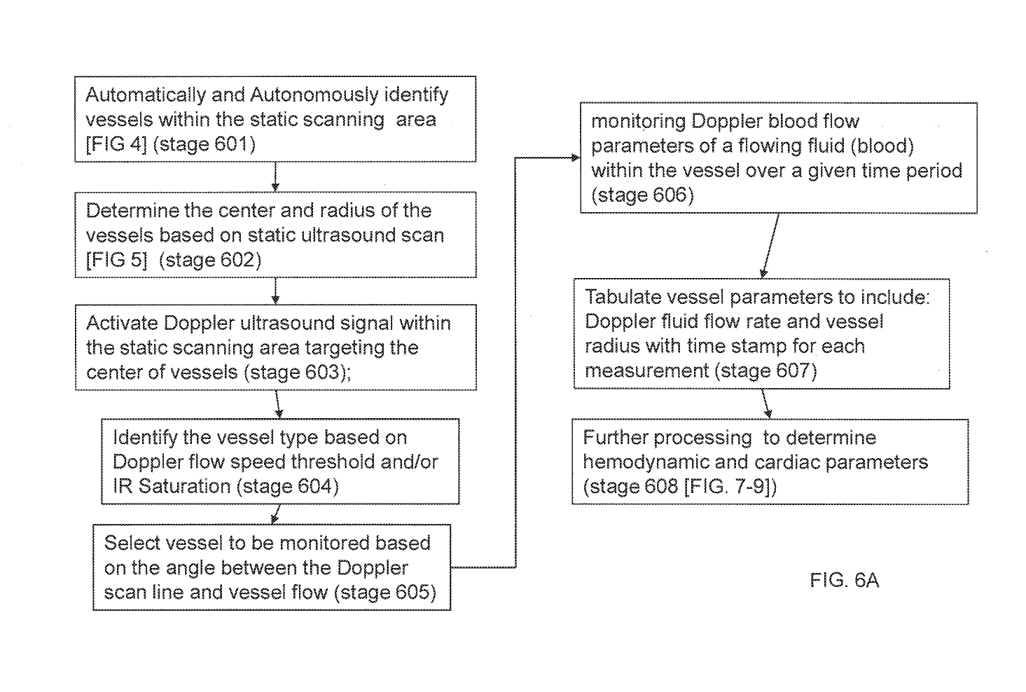

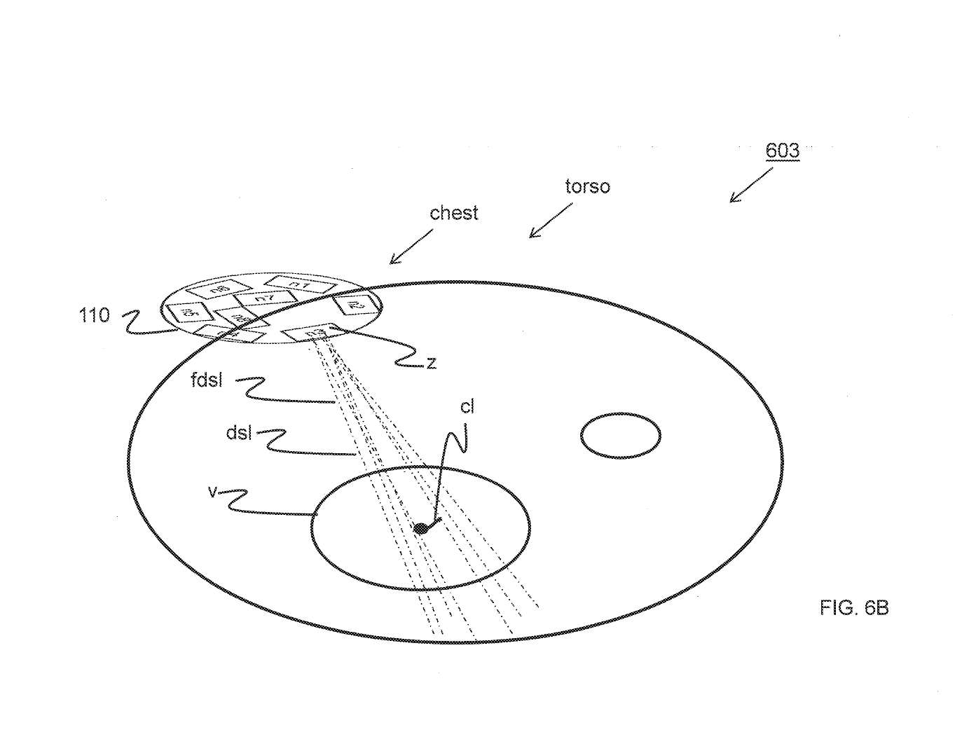

Termination of the combination of ultrasound (FIG. 4A-4C) and Doppler ultrasound (FIG. 6A-6B) scans provided with probe 110 initiates stage 302, where system 100 automatically and autonomously determines at least two vessel properties associated with at least one or more vessels detected within the underlying static scanning area. More detail describing the method for determining at least two or more vessel properties is provided in more detail in the flowchart of FIG. 4A and FIG. 5A. The vessel properties, determined for at least one or more vessels underlying the static scanning area, comprises vessel radius and vessel blood velocity with time stamp.

Next, in stage 303, system 100 further processes the at least two or more vessel parameters to obtain and/or determine a plurality of hemodynamic and cardiac parameters. The further processing described in stage 303 is provided with system management processor 102, scan engine 112, and decision support system 120. Optionally, further processing may be performed with a decision support system 120 based on the at least two vessel parameters determined in stage 302.

In an alternative embodiment, hemodynamic and/or cardiac parameters may, for example, include, but are not limited to, stroke volume, stroke volume index, heart rate, cardiac output, cardiac index, systolic blood pressure, diastolic blood pressure, mean arterial pressure, cardiac power, cardiac index, stroke volume variation, total peripheral resistance, or the like.

Optionally, hemodynamic and/or cardiac parameters may be displayed and/or presented to a system operator, subject, health care provider, decision support system, auxiliary device, communication device or the like, for determining follow up action, for example, including, but not limited to, further monitoring, medical intervention, drug course treatment, or the like in accordance with standard medical practice and procedures in relation to the determined hemodynamic and/or cardiac parameters.

FIG. 4A shows a flowchart depicting, in more detail, stage 302 of FIG. 3 for the combination of ultrasound and Doppler ultrasound scans with probe 110 comprising a plurality of ultrasound transducers and wherein each ultrasound transducers comprises an array of ultrasound elements, for example, piezoelectric element and/or crystals. The various stages depicted in FIG. 4A, particularly stages 404 and 405, are schematically illustrated in FIG. 4C.

Although the foregoing description describes ultrasound probe 110 with eight ultrasound transducers, each comprising at least 48 ultrasound elements, the present disclosure for a system and method for determining cardiac and hemodynamic parameters is not limited to such a probe, as system and method of the present application may be adapted to work with any multi-transducer ultrasound probe capable of producing a plurality of ultrasound scan-lines, for example, including, but not limited to, phased array, linear scan.

The method starts with stage 401 where the multi-transducer ultrasound probe is activated over the static scanning area producing an ultrasound signal of up to about 5 MHz, and more particularly, 2.5 MHz.

The ultrasound signal produced with probe 110 is controlled with scan engine 112 effectively to generate, transmit, and propagate the required ultrasound signal to the tissue underlying probe 110 within the static scanning area. Probe 110 provides a phased array ultrasound signal or, optionally, a continuous ultrasound signal. Next, the generated ultrasound signals are reflected back to probe 110, and the reflected ultrasound signal is detected with the (n) ultrasound elements of probe 110, and the reflected data is optionally processed in stage 402b and/or stored for offline processing in stage 402a. Optionally, some data may be processed online essentially in real time in stage 402b while other data may be processed offline in stage 402a. Optionally, initial processing may be provided essentially in real time in stage 402b and later completed offline in stage 402a.

Next, in stage 403, the initial ultrasound scan reflection data undergoes a low resolution mask detection, as is known and accepted in the art, to identify at least one or more potential vessels of interest underlying the static scanning area, for example, including, but not limited to, the aorta, pulmonary artery or the like vessels. If more than one vessel of interest is identified, then the method continues with respect to only one such vessel, where the remaining identified vessels will be processed in turn, optionally, sequentially or in a hierarchical manner, or the like graded manner.

Illustratively, low resolution mask detection is utilized in stage 403 so as to optimize the time taken to perform the scan with a plurality of ultrasound transducers of probe 110.

In the illustrative embodiment, low resolution mask detection is performed with each of the ultrasound transducers available with the multi-transducer probe 110. For example, an optional probe 110 comprising 4 ultrasound transducers, would perform a scan and low resolution mask detection of stages 401 and 403 with each of the 4 ultrasound transducers. For example, an optional probe 110 comprising at least two or more ultrasound transducers, for example, (n) ultrasound transducers would perform scan and low resolution mask detection of stages 401 and 403 with each of the (n) ultrasound transducers.

A first vessel of interest is identified according to optional vessel characteristics or criteria, for example, including, but not limited to, vessel size, vessel diameter, fluid dynamics through the vessel, angle formed between a transducer and vessel, or the like. For example, the vessel of interest may be a large vessel, such as the aorta and/or the pulmonary artery, such that it is identified based on a diameter of at least about 12 mm and optionally, from about 12 mm (millimeters) to about 42 mm (millimeters).

Next in stage 404, schematically illustrated in FIG. 4B, following low resolution mask detection, system 100 identifies and/or otherwise determines a subset (n−x) of the plurality (n) of the ultrasound transducer from the multi-transducer probe that provide for the best vessel criteria. Optionally, stage 404 is provided autonomously and automatically without the presentation of ultrasound imagery to a system operator. Vessel criteria may comprise vessel size, for example, diameter. More particularly, a vessel having a diameter of 12 mm or more is considered.

For example, system 100 utilizing a probe 110 comprising at least two or more (n) ultrasound transducers, for example, 8 ultrasound transducers, that may identify a subset (n−x) of the at least two or more transducers, for example, 3 or 4 transducers, that provide data in accordance with the vessel criteria. For example, the subset of ultrasound transducers selected may be selected in accordance with the vessel diameter of the identified diameter, for example, a diameter of at least 12 mm.

FIG. 4C provides a schematic non limiting illustration of stages 404 and 405 ending when the subset of ultrasound transducers (n−x) are selected and probe 110 rescans the static scanning area only with the determined subset of ultrasound transducers (n−x). Optionally, system 100 provides for saving both time and computational resources during the subset rescan by targeting the identified vessel of interest. Optionally, scan engine 120 controlling probe 110 provides additional control by controlling individual ultrasound scan lines.

More particularly, during low resolution mask detection, the borders of the vessel of interest are identified so as to allow scan engine 120 to determine which of the ultrasound transducers provided the relevant data based on the size of the detected vessel having a diameter of at least about 16 mm (millimeters). Optionally, with the identification of the vessel borders, scan engine 120 activates and/or generates an ultrasound signal utilizing the ultrasound scan-lines in the vicinity of the identified subset of transducers to concentrate the scan area about the vessel borders.

For example, scan engine 120 initially utilizing about 128, or more, scan-lines via probe 110, identifies that a vessel of interest, based on the size of the vessel having a diameter of at least 12 mm, is located between ultrasound scan-lines. Alternatively, scan engine 120 may utilize 15-92 scan lines via probe 110. These 15-92 scan lines were, therefore, identified as including the borders of a vessel of interest. Accordingly, upon rescan with a subset of ultrasound transducers, scan engine 120 activates and/or utilizes ultrasound scan-lines surrounding these 15-92 scan-lines, for example, 7-100 ultrasound scan-lines, so as to encompass the vessel of interest and account for vessel movement due to blood flow, stretch, breathing or the like, while, optionally, simultaneously saving resources during the subsequent scan and analysis.

Next, optional stage 407 is performed, following mask detection of stage 403 and, optionally, simultaneously with stage 404, to facilitate identification of a vessel of interest, if probe 110 comprises at least one or more infrared (herein referred to as ‘IR’) sensors. Illustratively, at least one or more IR sensors may be disposed with probe 110 to provide for and determine blood saturation in order to facilitate identifying at least one or more vessels of interest. For example, if the vessel characteristics and/or criteria utilize by the system and method of the present disclosure comprises blood saturation, the IR sensor may be utilized in conjunction with the ultrasound probe.

Next, in stage 405, schematically illustrated in FIG. 4C, following the rescanning of the static scanning area with a subset number of ultrasound transducers (n−x), for example, n−x of (n) transducers, a high resolution mask detection is undertaken to reevaluate, select, determine and/or otherwise identify an optimal transducer (z). The optimal transducer (z) from the plurality of (n) ultrasound transducers of probe 110 is selected based on parameters, for example, including, but not limited to, angle formed with the vessel of interest. Optionally, the optimal transducer (z) is selected based on a transducer to vessel angle of up to 60 degrees, and more particularly, an angle of about 45 degrees.

For example, the optimal ultrasound transducer (z) may be selected in accordance with the angle formed with respect to the vessel of interest, having a diameter of at least 12 mm, or more. Optionally, optimal transducer (z) may be selected based on a transducer to vessel angle of up to 60 degrees, and more particularly, an angle of about 45 degrees. For example, system 100 utilizing a probe 110 comprising (n) ultrasound transducers identifies a subset of (n−x) transducers where the transducers form an angle of about 55 degrees, a second transducer forms an angle of about 25 degrees, and a third transducer forms and angle of about 40 degrees with a vessel underlying the static scanning area.

Next, in stage 406, once an optimal transducer (z) from a plurality of transducers (n, n−x) is determined in stage 405, the optimal transducer is utilized for generating a Doppler ultrasound signal about the static scanning area, as will be depicted in more detail in FIG. 6. Optionally, the Doppler ultrasound signal may produce an image that may be presented to a system operator, however, system 100 may operate autonomously without system operator intervention and, therefore, may operate without generating or producing an image of the static scanned area.

FIG. 4B shows a flowchart of an optional method according to an optional embodiment of the present disclosure, similar to that depicted in FIG. 4A, however, the method performed such that the optimal transducer may be identified without performing stages 404 and 405. The method of FIG. 4B, therefore, identifies optimal transducer (z) utilizing a single scan mask, optionally using a low resolution scan mask a medium resolution scan mask, and a high resolution scan mask. The method according to FIG. 4B reduces the number of scans required to determine the optimal transducer (z) without rescanning a subset of transducers (n−x), as shown in FIG. 4A.

FIG. 5A shows a flow chart of a method according to the present disclosure for determining the center of a vessel of interest (v) within the static scanning area. The vessels identified during the ultrasound scan of the static scanning area (FIG. 4) produce an elliptical vessel surface, however, in order to determine cardiac and/or hemodynamic parameters associated with the vessel of interest the elliptical vessel surface must be converted to a tubular surface to identify the contour of the vessel of interest (v), that will thereafter provide for determining the hemodynamic and/or cardiac parameters, where the center of the vessel of interest (vc) and radius (vr) may be determined. The various stages depicted in FIG. 5A are schematically illustrated in FIG. 5B,

The method for converting the elliptical vessel surface sensed with the ultrasound probe 110 to a tubular vessel surfaces initiates with stage 501 where ultrasound reflection signals sensed with the ultrasound elements disposed in probe 110 corresponding to the ultrasound reflection from the vessel of interest within the static scanning area are converted to a plurality of points corresponding to the ultrasound reflections from the vessel surface.

Next in stage 502, the plurality of points corresponding to the surface of the vessel of interest undergo scan conversion, either low resolution mask detection as in stage 403 or high resolution scan conversion as described in stage 406 both of FIG. 4A. During mask detection the points are plotted on two axes.

Next, in stage 503, all points are confined within a quadrilateral about the two axes, where the quadrilateral structure formed houses and/or encompasses the surface of the vessel of interest.

Next, in stage 504, the coordinates for the quadrilateral center are identified by projecting at least two diagonals from each of the quadrilateral corners, where the center is determined to be the point of diagonal intersection.

Next, in stage 505, the quadrilateral center (vc) is utilized to project a plurality of chords, optionally, a chord is projected every 0.25 degree to produce 1440 chords about the quadrilateral center (vc) to intersect the surface of the vessel of interest (v). Optionally, a plurality of chords may be projected at least every 0.25 degree, or more, to produce up to 1440 chords.

Next, in stage 506, a subset of the projected chords (from stage 505) that intersect with the reflection coordinates corresponding to the vessel surface (from stage 501) are selected. Of the subset of chords, the smallest projected chord is utilized to determine the radius of the vessel of interest (vr). Optionally, the radius and center coordinates is utilized to convert the elliptical surface to a tubular surface.

Finally, in stage 507, based on the center (vc) and radius (vr), coordinates are utilized to tabulate data that relates the projected chords that intersect with the vessel surface, to creating a conversion table including the center coordinates, radius, and chord to radius ratio. Optionally, the table may further comprise transducer number, center scan-line number, beginning scan-line, end scan-line number, angle and depth. The conversion table may be utilized to identify the vessel of interest (v) surface utilizing polar point conversion.

Optionally, determination of the vessel center (vc) and vessel radius (vr), as depicted and described with respect to the flowchart of FIG. 5A, may be based on data obtained from each of (n) ultrasound transducers 114 of probe 110 and based on data obtained from at least a subset of transducers (n−x) and based on data from at least the optimal transducer (z).

FIG. 6A is an exemplary method according to the present disclosure depicting the use of the Doppler ultrasound to determine the blood flow velocity of the vessel of interest. As previously described in FIG. 4A, in stage 406 a Doppler ultrasound signal is utilized following a sequence of ultrasound signals and following the determination of the center and radius of the vessel of interest, as described in FIG. 5A.

The method for determining the blood flow vector through the vessel of interest initiates in stage 601, where a vessel of interest underlying the static scanning area is identified and targeted, as described in FIG. 4A-4B. Next, in stage 602, and as described in FIG. 5A, the center and radius of the vessel of interest is identified based on ultrasound reflection signals. Next, in stage 603, schematically illustrated in FIG. 6B, optionally, the optimal transducer (z) of probe 110, identified in stage 406 as described in FIG. 4A, is activated to produce a Doppler ultrasound signal over the static scanning area, targeting at least one or more vessel of interest. The optimal transducer may be activated such that it targets the center of at least one or more vessel of interest. The center of at least one or more vessel of interest is targeted by scan engine 120 to selectively activate a plurality of Doppler ultrasound scan-lines encompassing the center of a vessel of interest, as identified in stage 504 of FIG. 5A. The Doppler ultrasound scan-lines target the center of a vessel of interest by activating a plurality of flanking Doppler ultrasound scan-lines. Optionally, the flanking Doppler ultrasound scan-lines (fdsl) evenly flank the vessel center (vc) from each side. Optionally, the number of flanking scan-lines that are activated may be a function of a subject's data and/or vessel properties for example including but not limited to diameter, pulse rate, subject age, or the like. Optionally, the number and location of activated flanking scan-lines (fdsl) may be a function of a chord length (cl) or arc-length running through the vessel center or the vessel surface, as shown in FIG. 6B. Optionally, the length of the chord or arc (cl) associated with the center or vessel surface may be predetermined for example from about 0.3 mm to about 1.5 mm, and more particularly, 1 mm (one millimeter) or, optionally, may be a function of the radius, or the like, vessel properties or subject parameters.

For example a vessel having a diameter of 17 mm may produce 9 flanking Doppler scan lines comprising one Doppler scan line (dsl) going through the center and 4 flanking Doppler scan lines (fdsl) on either side of the center, such that scan lines are separated by a chord length (cl) of 1 mm.

For example, at least 2 or more, illustratively 7, Doppler ultrasound flanking scan-lines (fdsl), may be utilized to flank a vessel center (vc) where, for example, 3 flanking Doppler ultrasound scan-lines (fdsl) are activated on each side of the Doppler ultrasound scan-line that corresponds to the vessel center (vc). Such a Doppler scanning scheme provides for saving resources in particular time as ultrasound transducers and elements are selectively activated, and furthermore provide for real time computational constraints. Flanking the vessel center further provides for ensuring that vessel movement due to breathing, stretching and the like motion are accounted for while encompassing the center of the vessel blood flow. Optionally, each flanking scan-line (fdsl) may be generated at a set interval from one another such that a first one is fired at t=0 and the next fdsl is generated at a set time interval t=x. Optionally, the inter flanking scan line time interval may, for example, be from about 2 ms (millisecond) up to about 15 ms (millisecond), and more particularly, about 2.5 ms (millisecond), or any time interval delay for example in a resolution of about 0.1 ms (millisecond) from about 2 ms up to about 15 ms (millisecond). Optionally, the inter flanking scan lines time may be controllable and determined based on vessel parameters, user parameters, physician parameters, or the like.

Optionally, the Doppler ultrasound signal generated during stage 603 may be used to validated and confirm the vessel parameters determined with ultrasound scan to reconfirm the radius determined with ultrasound scans, as determined in stage 506 of FIG. 5A.

Optionally, in stage 604 the vessels of interest (v) may be categorized and/or identified based on fluid dynamics of the blood flowing through the vessel. Illustratively, the vessel of interest may also be identified based on IR saturation data optionally provided in stage 407 of FIGS. 4A-4B. Optionally, the vessel of interest (v) may be identified based on at least one or both of on fluid dynamics of the blood flowing through the vessel and/or IR saturation data. For example, identification of fluid dynamics corresponding to laminar blood flow is indicative of the pulmonary artery. For example, identification of fluid dynamics corresponding to turbulent blood flow is indicative of the aorta. Fluid dynamics corresponding to blood flow velocity is utilized to identify the vessel type. Optionally, the determination of vessel type is based on a threshold blood flow velocity. Optionally, the threshold blood flow velocity may be about 60 cm per second (60 cm/sec). For example blood flow below a threshold velocity of about 60 cm/sec identifies the vessel as the pulmonary artery (PA). For example, blood flow above a threshold velocity of about 60 cm/sec identifies the vessel as the aorta (AO).

Next, in stage 605, the angle formed between the vessels of interest and the ultrasound transducer, specifically the optimal transducer (z), is determined. In one embodiment of the present disclosure, one vessel to be monitored is selected from the vessels of interest based on the angle formed with the ultrasound transducer. Optionally, the angle formed is from about 20 degrees to about 60 degrees, and more particularly the selected vessel is selected based on the vessel that forms an angle closest to 45 degrees.

Next, in stage 606, the Doppler ultrasound about the individual flanking Doppler ultrasound scan lines (fdsl) are activated and monitors the vessel to be monitored (v), determined in stage 605, for a given period corresponding for example up to about 6 s (six seconds) to allot for measuring a Doppler echocardiogram at least 4 cardiac cycles. Such monitoring provides for determining blood flow velocity through the vessel (v).

Optionally, radius parameters obtained from individual Doppler scan lines (dsl), comprising a plurality of flanking scan lines (fdsl), are cross referenced against the radius obtained from the ultrasound scan of stage 506. Optionally, parameters obtained and/or otherwise determined based on individual Doppler scan lines (dsl), including a plurality of flanking Doppler scan lines (fdsl), FIG. 6B, may be used to cross reference and validate vessel parameters obtained from individual Doppler scan lines (dsl). Optionally, a Doppler scan lines (dsl) validation table may for example be determined based on triangulation calculation relating to parameters for example including but not limited to dsl length, dsl-angle, vessel radius, dsl-angle, cl-chord length distance, or the like. Optionally, the Doppler scan line validation table provides for validating vessel radius.

Next, in stage 607, the two vessel properties including vessel radius and blood flow speed each with a time stamp are tabulated and stored in system 100. Next, in stage 608, the three vessel properties identified undergo further processing with the system management processor 102 to determining a plurality of cardiac and/or hemodynamic parameters.

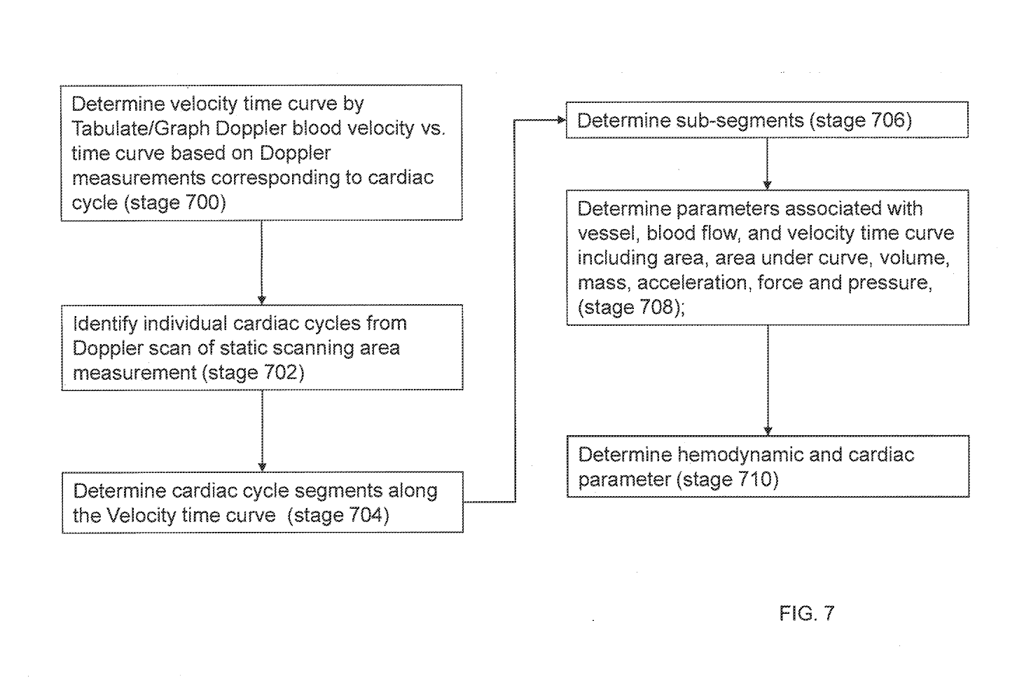

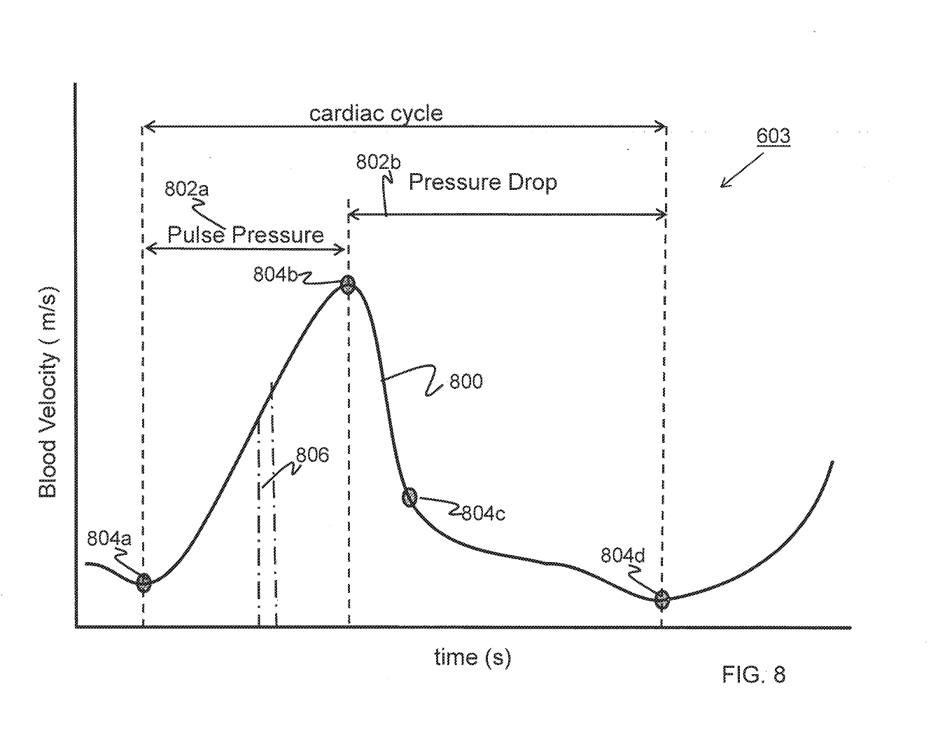

FIG. 7 is a flowchart of an exemplary method according to an optional embodiment of the present disclosure for determining hemodynamic and cardiac parameters automatically based on Doppler ultrasound parameters, defining an optional method for further processing of the Doppler ultrasound parameters and vessel parameters identified and/or measured within the static scanning area, as described and depicted herein above. The method initiates in stage 700 wherein the Velocity time curve data is tabulate, and/or optionally presented in the form of a plotted graph (800 FIG. 8), in the form Doppler blood velocity (s=meter per second) vs. time curve (t=seconds), as schematically shown in FIG. 8. Stage 700 is optionally performed based on the data measured, tabulated and/or otherwise provided in stage 606 and 607, depicted and described in FIG. 6A. Optionally, the Velocity time curve data presented corresponds to at least one or more cardiac cycles. Optionally, the Velocity-Time curve data presented corresponds to at least four or more cardiac cycles, and alternatively, at least three or more consecutive cardiac cycles.

Optionally, the method in any one or more of its stages may be performed manually and/or automatically and/or semi-automatically or in any combination thereof. Optionally, the method described may be performed by a user, for example, including, but not limited to, the subject, a healthcare giver, physician, technician or the like trained individual. The scan may be performed automatically and autonomously without user intervention with system 100 and with system management processor 102. Optionally, the manual and/or automatic performance of the method may be provided with a remote system for example including but not limited to decision support system 120. Medical decision support system 120 may for example be provided in the form of a back office and/or call center, and/or health care provider, and/or ambulatory care, and/or telemedicine center, and/or a medical decision support system, and/or a caregiver decision support system.

Next, in stage 702, processing of the Velocity-Time curve data is initiated by identifying and/or determining at least one or more of the individual cardiac cycles measured in stage 606, from at least three or more consecutive cardiac cycles. An exemplary Velocity-Time curve 800 is schematically illustrated in FIG. 8, showing a cardiac cycle from point 804a to 804d, optionally defining the diastolic points about the Velocity-Time curve 800.

Next, in stage 704, the Velocity-Time curve data corresponding to the individual cardiac cycles identified in stage 702 is segmented into the plurality of cardiac cycle segments 802, which are identified within each of the available cardiac cycles, as shown in FIG. 8. Optionally, at least one or more cardiac cycle segments 802a, b may be identified. Illustratively, at least two cardiac cycle segments are identified within each available cardiac cycle data, for example, cardiac cycle segments 802a corresponding to pulse pressure and 802b corresponding to pressure drop, as shown in FIG. 8.

Optionally, the cardiac cycle segmentation may be performed manually by a user, for example, including, but not limited to, the subject, a physician, a trained technician, or the like. Optionally, the number of cardiac cycle segments identified, and/or the cardiac cycle segmentation resolution may be controlled based on parameters, for example, system 100 dependent parameters, parameters independent of system 100, parameters measured with system 100, external parameters, and/or controllable parameters, or any combination thereof.

Optionally, the segmentation is performed based on known cardiac cycle landmarks, for example, including but not limited to diastole, systole, isovolumic contraction, isovolumic relaxation, A-V valve closure, aortic valve opening, ejection, A-V valve opening, or the like.

In an alternative embodiment, cardiac cycle segmentation and cardiac landmark identification may be performed automatically by mathematical manipulation of the Velocity-Time curve to identify extrema about the curve and/or within the tabulated data. Velocity-Time curve extrema may, for example, include, but is not limited to, identifying maximum, minimum, local maximum, local minimum, absolute maximum, absolute minimum, inflection points, gradient, slope, supremum, infimum, or the like. An example of such extrema points 804a-d is schematically illustrated in FIG. 8. For example, point 804a schematically illustrates the point corresponding to the start of a cardiac cycle and associated with the diastole while point 804d corresponds to the end of a cardiac cycle also associated with the diastole; point 804b optionally corresponds to the maximum velocity and the end of the pulse pressure 802a, during the cardiac cycle, point 804c corresponds to the inflection point during pressure drop segment 802b.

Next, in stage 706, individual cardiac cycle segments are further subdivided into a plurality of (i) sub-segments, as schematically illustrated in sub-segments 806 in FIG. 8. Optionally, the resolution and/or the number of sub-segments may be constant, time based and/or based on the resolution of the Velocity-Time curve, user defined or the like.

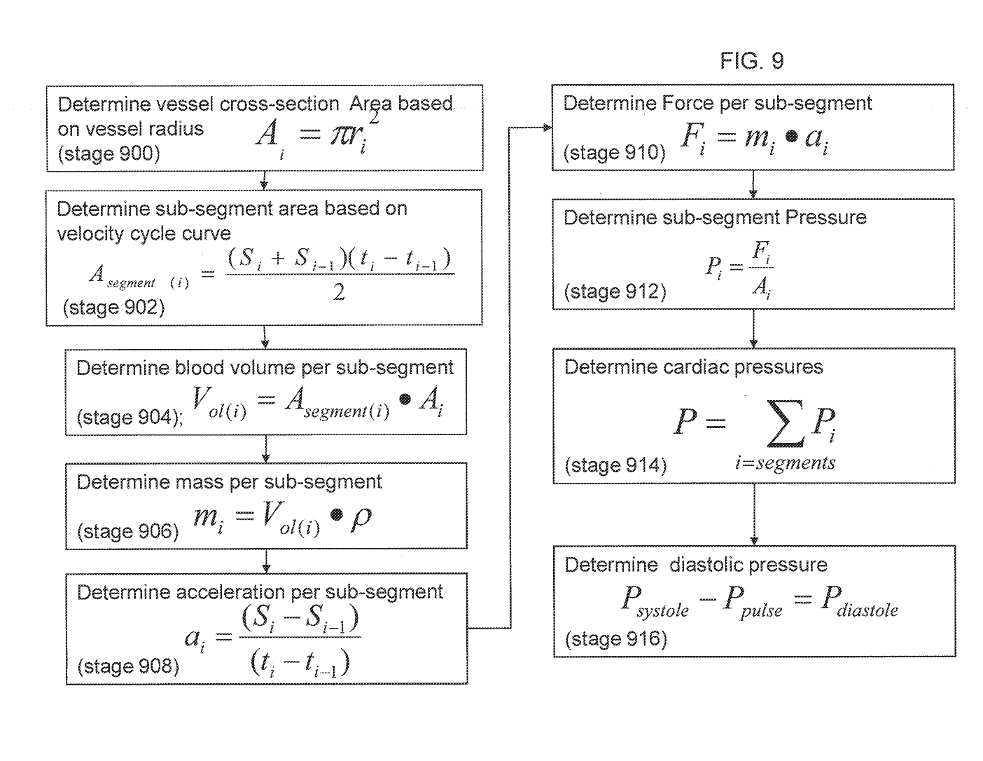

Next, following segmentation and sub-segmentation during stage 708, a plurality of vessel associated parameters are determined and/or otherwise calculated that are optionally based on vessel parameters, Doppler blood flow parameters measured and/or otherwise determined with system 100 of the present disclosure as described hereinabove. Optionally, the parameters determined may, for example, include but are not limited to, area, area under the Velocity-Time curve, area of cardiac cycle segment, area of cardiac cycle sub-segment, vessel volume, blood flow mass, blood flow acceleration, force and pressure, as depicted in FIG. 9 below.

Next, in stage 710, hemodynamic and cardiac parameters are determined based on parameters determined and/or otherwise identified in stage 708 that may, for example, include but are not limited to, stroke volume, stroke volume index, heart rate, cardiac output, cardiac index, systolic blood pressure, diastolic blood pressure, mean arterial pressure, cardiac power, cardiac index, stroke volume variation, total peripheral resistance, or the like.

### Example 1

### Determination of Diastolic Pressure in Aorta

The foregoing example is provided for illustrative purpose only and does not limit the present application to the description or calculation as described as set forth.

Diastolic pressure of the aorta, for example, may be determined in a non-invasive manner based on a combination of ultrasound and Doppler scan of a static area about a subject's upper torso, for example, a static scanning area about the subject's chest, as described and depicted herein above in FIGS. 1-8. Optionally, the determination below is performed for individual cardiac cycles measured with the Doppler ultrasound about the static scanning area targeting at least one vessel of interes, for example, the aorta or pulmonary artery.

In stage 900, the vessel's cross-section area is determined based on the vessel radius as determined according to the method described hereinabove and based on Equation 1 below, for each sub-segment 806 (i).

Aᵢ=πrᵢ² EQ1.

Next, in stage 902, the area of at least one, and more particularly, a plurality of sub-segments (i), for example, 806 of FIG. 8, is determined from the Velocity-Time curve 800 of FIG. 8, or from the corresponding tabulated data, according Equation 2 below. The sub-segment area of each sub-segment 806 is determined for the full curve 800; therein accounting for the full time range of is Doppler scan measurement.

$${A}_{segment ( i )} = \frac{( {S}_{i} + {S}_{i - 1} ) ( {t}_{i} - {t}_{i - 1} )}{2} . \qquad EQ 2$$

Next, in stage 904, the blood volume is determined per sub-segment (i) 806 according to Equation 3.

Vₒₗ₍ᵢ₎=A_{segment(i)}·Aᵢ EQ3.

Next, in stage 906, the blood mass per sub-segment (i) 806 is determined based the volume determined in Equation 3 and a constant density (ρ).

mᵢ=Vₒₗ₍ᵢ₎·ρ EQ4.

Next, the stage 908, blood acceleration per sub-segment (i) 806 is determined based on curve 800 as determined in Equation 5.

$${a}_{i} = \frac{( {S}_{i} - {S}_{i - 1} )}{( {t}_{i} - {t}_{i - 1} )} . \qquad EQ 5$$

Next, in stage 910, the force per sub-segment (i) 806 is determined based on Equation 6.

Fᵢ=mᵢ·aᵢ EQ6.

Next, in stage 912, the pressure per sub-segment (i) 806 determined based on Equation 7.

$${P}_{i} = \frac{{F}_{i}}{{A}_{i}} . \qquad EQ 7$$

Next, in stage 914, segment pressures (802a, b) are determined for each segments, respectively accounting for pulse pressure (Pₚᵤₗₛₑ) segment 802a and the pressure of pressure drop (P_{drop}) segment 802b, and the total pressure (P_{systole}) experienced during the cardiac cycle as defined from points 804a to 804d. The total pressure and the segment pressure is determined by summing the relevant and individual sub-segments 806, for example, as shown by Equation 8. Optionally, the total pressure is equivalent to the systolic pressure (P_{systole}) of individual cardiac cycles measured with the Doppler ultrasound scan of the static scanning according to optional embodiment of the present disclosure. Optionally, the total pressure may be determined by summing pulse pressure (Pₚᵤₗₛₑ) and pressure drop (P_{drop})

Next, in stage 916, the segment pressures determined in stages 914 are utilized to determined the diastolic pressure experienced in the measured vessel of interest (v), according to Equation 9 below, of the measured vessel.

P_{systole}−Pₚᵤₗₛₑ=P_{diastole} EQ9.

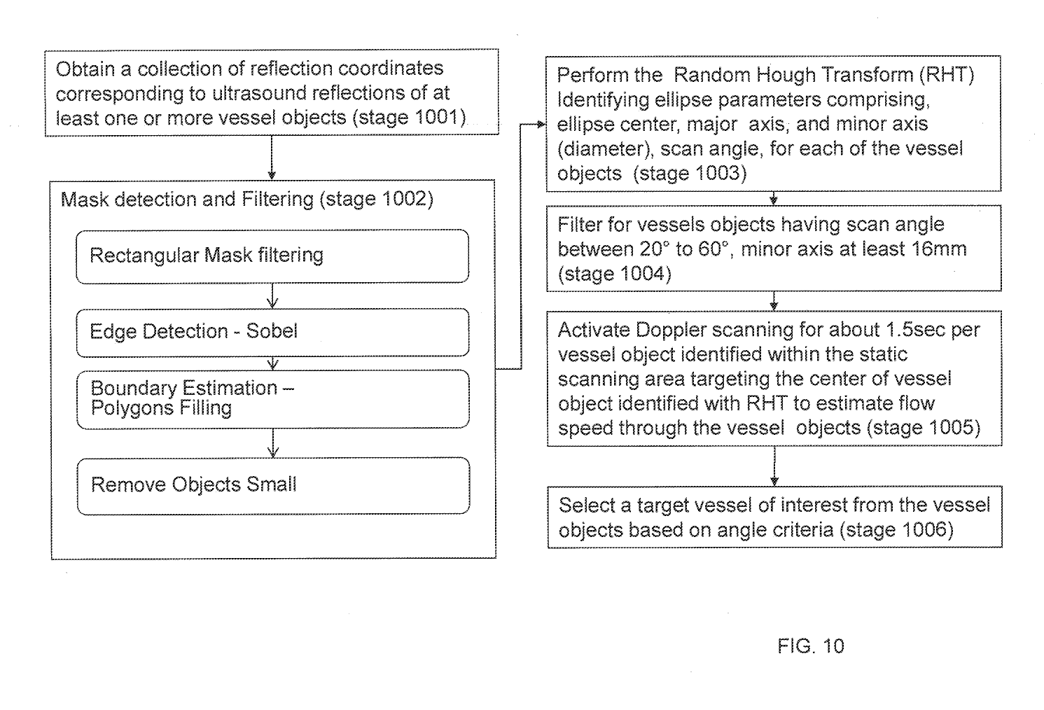

Now referring to FIGS. 10-12, describing an optional embodiment of the present disclosure provided for determining hemodynamic and cardiac parameters based on a static scanning using a combination of ultrasound and Doppler scans.

FIG. 10 shows a flow chart describing an optional method according to the present disclosure provides for identifying a vessel of interest and obtaining cardiac parameters associated with the vessel for further processing to determining cardiac and hemodynamic parameters.

First in stage 1001 a plurality of scanned objects are automatically and autonomously identified within the static scanning area by the initial ultrasound scan for example as previously described in details with respect to FIG. 4.

Next in stage 1002 mask detection and filtering is performed to identify a first of potential set of vessel of interest from within the plurality of scanned objects. Optionally and preferably the filtering process may for example include at least one and more preferably plurality of filters selected from the group including but not limited to rectangular mask filtering, edge detection, boundary estimation, object shape and/or size threshold, or the like alone or any combination thereof. Optionally edge detection may for example be provided in the form of Sobel edge detection. Optionally Boundary estimate may be provided with polygon filling. Optionally object size threshold may for example include removing objects that are too small based on size estimation, shape estimation for example remove is not ellipse or circular, or any combination thereof.

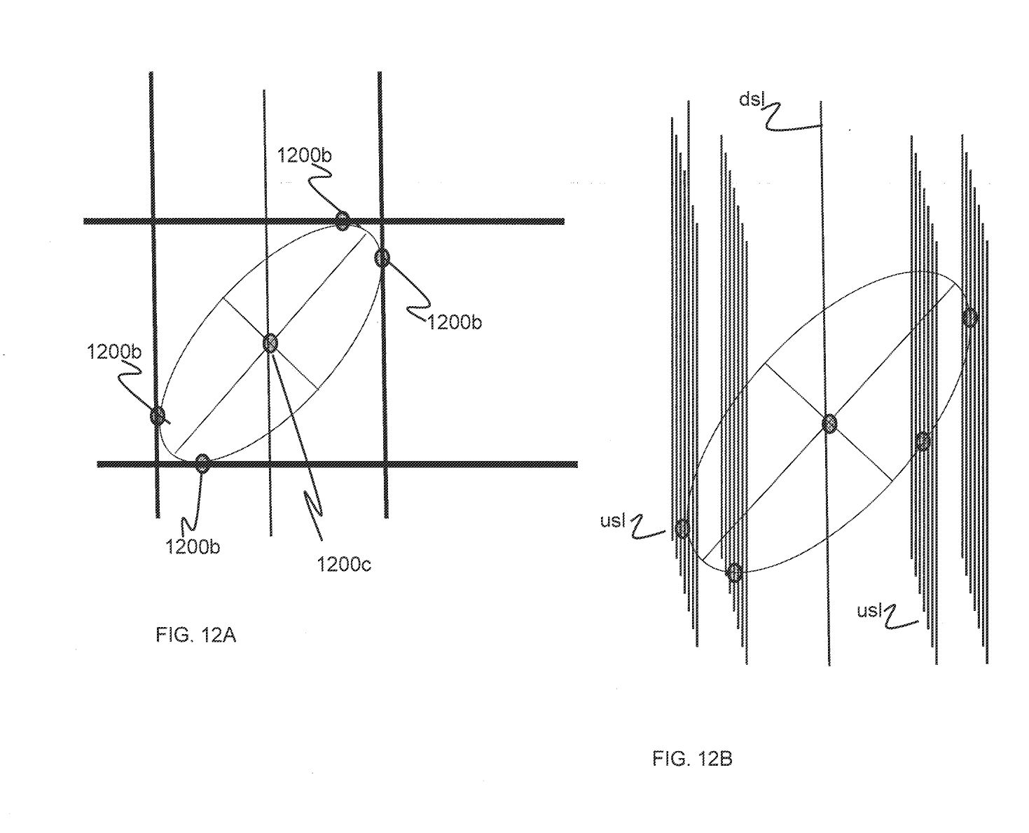

Next in stage 1003 the filtered vessel object, most preferably in the form of ellipse, ellipsoid, or circle or the like rounded closed formation, identified in stage 1002 undergo a transformation, for example including but not limited to Random Hough Transform, to further identify vessels of interest for example including but not limited to the aorta and pulmonary artery. One transformation acceptable for use is the Random Hough Transform (herein referred to ‘RHT’) to identifying parameters associated with the vessel objects identified. In one embodiment, the RHT is preformed for an ellipse, as it is assumed that any vessel of interest identified in stage 1002 will have an elliptical profile. Utilization of RHT provides for direct and/or indirect identifying and/or inferring a plurality of parameters associated with the vessel for example including but not limited to ellipse center, major axis, and minor axis and scan angle. For example as illustrated in FIGS. 12A-B, showing the axis as well as the ellipse boundary points.

Next in stage 1004 the RHT parameters are utilized to filter for a subset of vessels for further scanning to identify the vessel of interests selected from the aorta (AO) and/or pulmonary artery (PA). The filtering criteria may comprise a scan angle from about 20 degrees to 60 degrees and a minor axis of at least 16 mm and up to 40 mm (millimeters). Optionally the diameter of the vessel of interest is correlated with and/or otherwise associated as a function of the minor axis, determined from the RHT processing. Optionally the diameter of the vessel of interest may be correlated with and/or otherwise associated as a function of the major axis, determined from the RHT processing. Optionally the diameter of the vessel of interest is correlated with and/or otherwise associated as a function of both the major and minor axes, determined from the RHT processing.

Next in stage 1005 a further filtering process is performed to identify at least one vessel of interest selected form the aorta (AO) or pulmonary artery (PA) by utilizing a Doppler scan, to determine the blood velocity flowing through the vessel of interest. At least one or more scan line(s) is directed to the vessel center identified by the RHT in stage 1003 for the vessels subset identified and filtered for in stage 1004. The Doppler scan provides for determining the blood flow velocity through the vessel of interest, most preferably about its center. Optionally the Doppler scan is performed from about 1 second to about 3 seconds, and most preferably for 1.5 seconds per each vessel of interest identified. Optionally, the Doppler scan is performed for at least one full cardiac cycle, preferably mapping the blood velocity fluctuation during the cardiac cycle. Optionally and preferably the Doppler scan provides to scan and measure the maximum blood flow velocity through the vessels center occurring during at least one cardiac cycle.

Finally in stage 1006 at least one or more vessel of interest is indentified by selecting the vessel relative to at least one threshold associated with the vessel(s) of interest, for example including but not limited to vessel blood flow velocity, and/or vessel diameter, the like or any combination thereof. Optionally the threshold values may be determined based on user parameters and/or subject parameters, and/or healthcare giver and/or system parameters. Optionally threshold values may be determined based on at least one or more parameter(s) for example including but not limited to state of health, medical history, age, medical conditions, cardiac history, anatomical parameters, atherosclerosis parameters, any combination thereof or the like.

Optionally the blood flow velocity threshold may be about 0.6 m/sec and may be indicative of the type of vessel being scanned. Optionally monitoring of the aorta is selected based on a blood flow velocity threshold that is above an optional threshold of 0.6 m/sec or the pulmonary artery is selected as having a blood flow velocity below the threshold of 0.6 m/sec. Optionally the vessel diameter threshold may for example be from about 16 mm to about 40 mm.

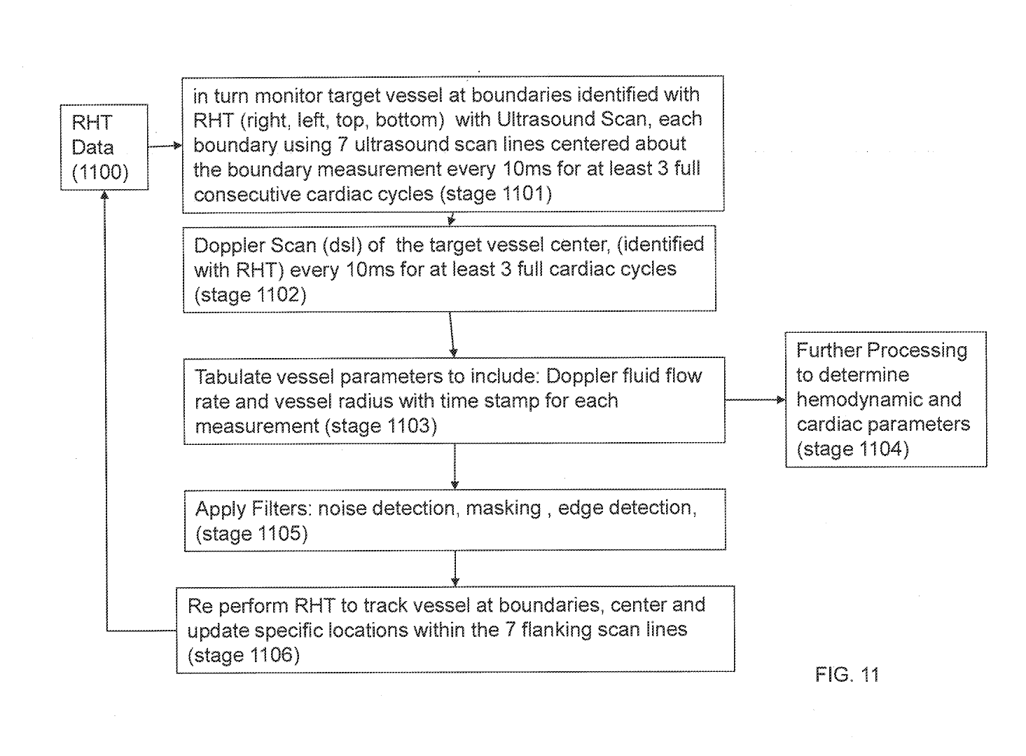

FIG. 11 depicts an optional embodiment of the present disclosure of a method for continuously monitoring vessel parameters of a vessel of interest identified and selected according to an optional embodiment of the present disclosure, for example the method described in FIG. 10. The method initiates in stage 1100 by identifying vessel properties as determined by the RHT transform as previously described in FIG. 10 in stage 1003 and as schematically illustrated in FIG. 12A.

Next in stage 1101 at least one or more vessel of interest, for example as identified in stage 1006 of FIG. 10, are processed by analyzing individual vessel assuming the vessel is represented by an ellipse, for example as illustrated in FIGS. 12A-B. Vessel boundary 1200b, data points obtained from the RHT processing, for example including but not limited to top, bottom, left, right boundary coordinates are identified and processed in turn. An ultrasound scan line is targeted at each coordinate 1200b, in turn, where each is flanked by a plurality of flanking scan, for example 7 ultrasound scan lines (usl) are directed at each boundary 1200b, FIG. 12B, coordinate comprising one scan line targeting the coordinate calculated with the previous scan, along with about three flanking scan lines flanking each side of the central scan lines, such that about seven ultrasound scan lines are centered about each boundary point 1200b. The flanking scan lines provide for identifying movement, expansion, deformation and changes of the vessel coordinates, over time and during stages of the cardiac cycle. The vessel ultrasound data, at the boundary 1200b, is determined every 10 milliseconds (10 ms) for about 6 seconds providing data for at least three or more consecutive cardiac cycles.

Next in stage 1102 the RHT identified vessel center 1200c, as shown in FIG. 12A-B, is similarly tracked with a Doppler scan line (dsl). In one embodiment, the vessel ultrasound data is determined every 10 ms for about 6 seconds providing data for identifying at least three or more consecutive cardiac cycles.

Next in stage 1103 data relating to vessel dimension via ultrasound boundary 1200b scanning and blood flow velocity via Doppler scanning of vessel center 1200c is tabulated with a time stamp, providing data every 10 ms. In one embodiment, in stage 1104 the tabulated data is then processed to further identify hemodynamic and cardiac parameters, for example including but not limited to diastolic pressure, systolic pressure, pulse pressure or the like. Optionally further processing is provided as taught by optional embodiments of the present disclosure shown in FIGS. 7-9 and 13-15.

Next in stages 1105 and 1106 ultrasound and Doppler data provided in earlier stages 1101 and 1102 is then processed further to track the vessel of interest to account for vessel movement, deformation or the like. Specifically, in stage 1105 at least one or more filters are applied to the ultrasound data and Doppler data, for example as described in FIG. 10, most preferably to include noise detection, masking, and edge detection for example to provide an up to date an accurate image and localization of the vessel of interest within the static scan area.

In stage 1106 the filtered data is then further processed utilizing the RHT, for example to redefine the location of boundary 1200b and center 1200c illustrated in FIG. 12A-B, to reanalyze and identify the vessel parameters for example including but not limited to vessel center, vessel diameter via ellipse minor axis, boundaries via major axis, and scan angle. The updated vessel RHT parameters are then preferably utilized in the following vessel scan, most preferably 10 ms following the initial scan, therein updating the RHT Data of stage 1300 most preferably about every 10 ms.

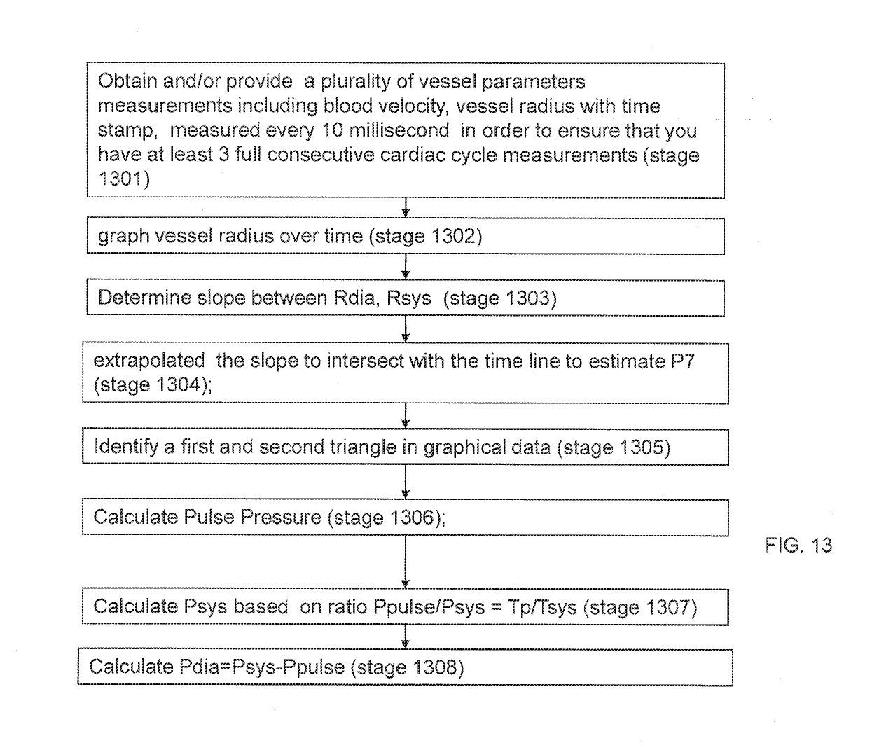

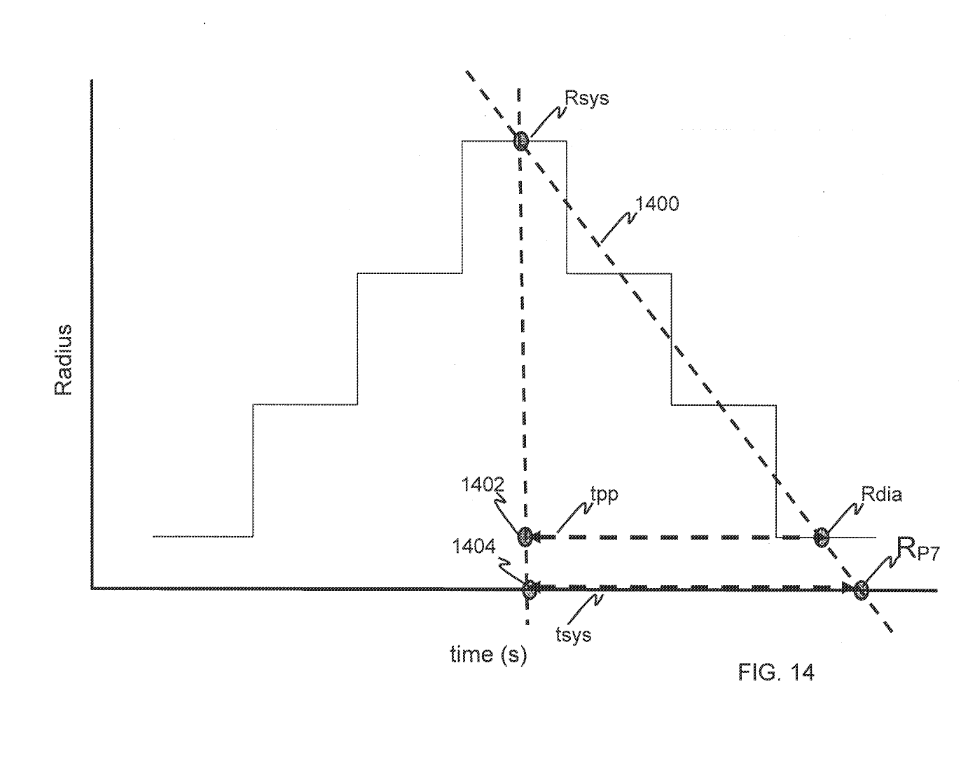

Now referring to FIGS. 13 and 14, describing an optional embodiment of the present disclosure provides a method for non-invasively determining cardiac parameters and in particular to a method for noninvasive determination of at least one and more preferably a plurality of cardiac cycle parameters for example including but not limited to Systolic Pressure (herein referred to as ‘Psys’), Diastolic Pressure (herein referred to as ‘Pdia’), and Pulse Pressure (herein referred to as ‘Ppulse’ and/or ‘PP’).

FIG. 13 shows an optional embodiment according to the present disclosure for determining cardiac and/or hemodynamic parameters based on measured data defining a blood flow velocity and vessel radius, for example as described in FIGS. 4-6 and/or 10-12. The method according to an optional embodiment of the present disclosure for further processing of vessel radius, and blood flow velocity, comprises, first stage 1301 providing a plurality of measurements for at least 3 or more consecutive cardiac cycles where optionally and preferably the measurements are calculated every 10 milliseconds (10 ms) for about 6 seconds, and tabulate results comprising vessel radius, blood flow velocity through vessel, for example as previously described in FIGS. 4-6 and/or FIGS. 10-12.

Stage 1302 the vessel radius is graphed as a function of time, for example as schematically illustrated in FIG. 14. In one embodiment, the maximum/peak radius is correlated with and/or otherwise a function of systolic radius, Rsys, and the diastolic radius, Rdia, is correlated with and/or otherwise a function of the minimum radius.

Stage 1303 a line, 1400, is extrapolated from the maximum radius, Rsys as shown in FIG. 14, through the minimum radius, Rdia, until line 1400 intersect the time line defining the point of intersection with the time line, provided to estimate the virtual radius R_{P7} as showing in FIG. 14, R_{P7} the vessel radius corresponds to the an ideal blood vessel radius (R_{P7}) where at pressure is at a minimum and equivalent to about 2 mmHg to about 10 mmHg and most preferably 7 mmHg.

Next in stage 1304 utilizing the extrapolated line 1400 determine a first triangle (Rsys, Rdia, 1402) correlating the pulse pressure defined wherein the hypotenuse corresponds to the slope between the systolic (Rsys) and diastolic radius (Rdia); and define a second triangle (Rsys, R_{P7}, 1404) defined wherein the hypotenuse corresponds to the slope between the systolic radius (Rsys) to the ideal radius at lowest pressure (R_{P7}).

Stage 1305 determine base of both triangles where tpp based on baseline of first triangle and tsys related to second triangle.

Next in stage 1306 pulse pressure (Ppulse) is determined, for example as described in FIG. 8, segment 802a, by optionally determining the energy of and/or the area under the curve of the blood velocity vs. time graph as previously described.

Next in stage 1307 evaluate ratio correlating the pulse pressure and systolic pressure with the pulse pressure time and systolic time as calculated above, stages 1305 and 1306, to determine the Systolic Pressure and give by the equation 10 (EQ10.) below:

$$\frac{Ppulse}{Psys} = \frac{tpp}{t s y s}$$

Most preferably tsys and tpp are provided from the first and second triangle, stage 1305, while Pressure Pulse, stage 1306, to determine:

$$Psys = Ppulse [ \frac{tsys}{tpp} ]$$

Finally in stage 1308 the Pdia is calculated from the Psys, and Ppulse data, based on the equation Pdia=Psys−Ppulse.

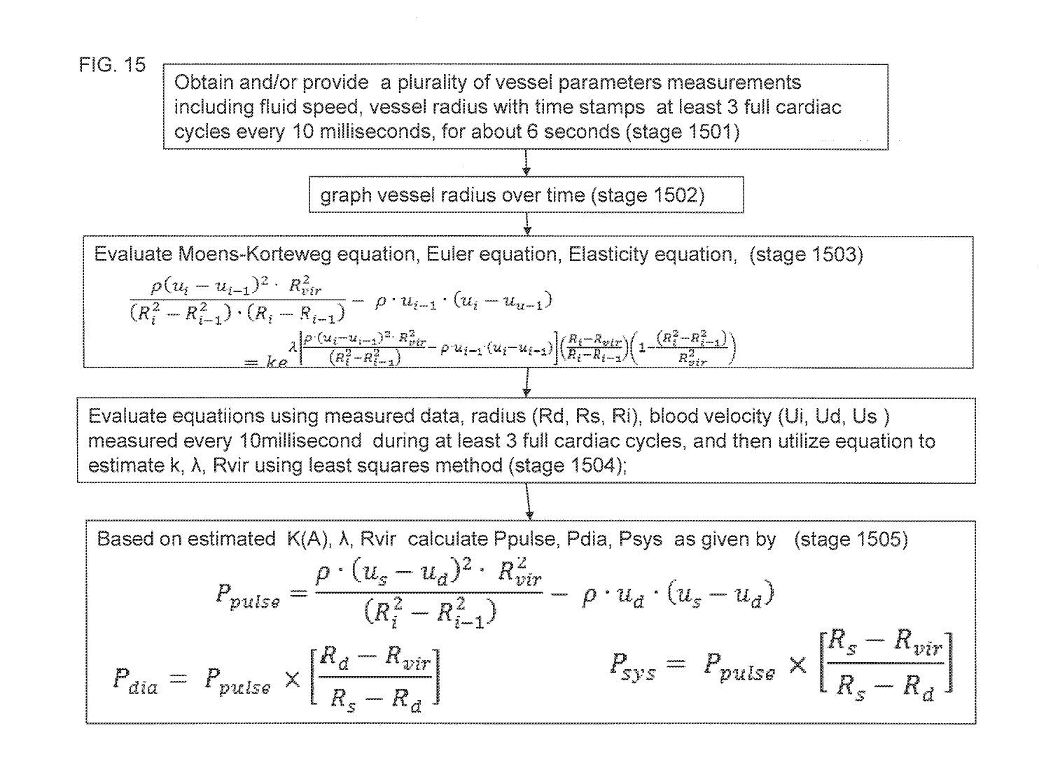

Now referring to FIG. 15, describes an optional embodiment of the present disclosure provides a method for non-invasively determining cardiac parameters and in particular to a method for noninvasive determination of at least one and more preferably a plurality of cardiac cycle parameters for example including but not limited to Systolic Pressure (herein referred to as ‘Psys’), Diastolic Pressure (herein referred to as ‘Pdia’), and Pulse Pressure (herein referred to as ‘Ppulse’ and/or ‘PP’). In one embodiment, Psys, Pdia, Ppulse are determined by manipulating a plurality of equations for example including but not limited at least one or more and/or a combination of the Moens-Korteweg equation, Euler's equation and generalized Hooke's law of Elasticity, coupled with a plurality of measurements relating vessel radius, vessel blood flow velocity and time stamp, as provided by exemplary embodiment of the present disclosure as described in FIGS. 4-6 and/or FIGS. 10-12. Optionally the vessel radius, blood flow velocity and time stamp, may be provided with an ultrasound and Doppler scanning device according to an exemplary embodiment of the present disclosure as described in FIGS. 1-3 according to the present disclosure. Optionally the vessel radius, and blood flow speed with time stamp may be provided by an external and/or auxiliary device.

FIG. 15 shows an optional embodiment according to the present disclosure for determining cardiac and/or hemodynamic parameters based on measured data defining a blood flow velocity and vessel radius, for example as described in FIGS. 4-6 and/or 10-12. The method according to the present disclosure comprises first stage 1501 providing a plurality of measurements for least three or more consecutive cardiac cycles where optionally the measurements are calculated every 10 milliseconds for about 6 seconds, and tabulate results comprising time stamp, vessel radius, blood flow velocity through vessel, for example as previously described in FIGS. 4-6 and/or FIGS. 10-12.

Next in stage 1502, the tabulated data is graphed to show the evolution of vessel radius over time and optionally the blood flow velocity over time.

Next in stage 1503, the data provided in stage 1501 is used to evaluate a plurality of vessel associated equations for example including but not limited to Moens-Korteweg equation (M), Euler's equation (E) and generalized Hooke's law of Elasticity (H). Most preferably a combined proprietary equation as provided below:

$$\frac{{ρ ( {u}_{i} - {u}_{i - 1} )}^{2} \cdot {R}_{vir}^{2}}{( {R}_{i}^{2} - {R}_{i - 1}^{2} ) \cdot ( {R}_{i} - {R}_{i - 1} )} - ρ \cdot {u}_{i - 1} \cdot ( {u}_{i} - {u}_{u - 1} ) = {ke}^{λ [ \frac{{ρ ( {u}_{i} - {u}_{i - 1} )}^{2} \cdot {R}_{vir}^{2}}{( {R}_{i}^{2} - {R}_{i - 1}^{2} ) \cdot ( {R}_{i} - {R}_{i - 1} )} - ρ \cdot {u}_{i - 1} \cdot ( {u}_{i} - {u}_{u - 1} ) ] ( \frac{{R}_{i} - {R}_{vir}}{{R}_{i} - {R}_{i - 1}} ) ( 1 - \frac{( {R}_{i}^{2} - {R}_{i - 1}^{2} )}{{R}_{vir}^{2}} )}$$

Next in stage 1504, the above equation is evaluated using the measured data, for example as provided by the device, system and method of optional embodiments of the present disclosure, as described in FIGS. 1-12, utilizing radius (Rd, Rs, Ri), blood flow velocity (Ui, Ud, Us) most preferably provided at about every 10 millisecond (10 ms) for at least 6 seconds to account for at least three or more consecutive cardiac cycles, and then utilize the resultant plurality of equations to estimate k, λ, Rvir using the least squares method.

Next in stage 1505 the estimated parameters including k, λ, Rvir are utilized to evaluate and/or calculate Psys, Pdia, Ppulse according to the equation below determined in stage 1501 and based on a plurality of vessel associated equations.

$${P}_{dia} = {P}_{pulse} \times [ \frac{{R}_{d} - {R}_{vir}}{{R}_{s} - {R}_{d}} ]$$

$${P}_{sys} = {P}_{pulse} \times [ \frac{{R}_{s} - {R}_{vir}}{{R}_{s} - {R}_{d}} ]$$

$${P}_{pulse} = \frac{ρ \cdot {( {u}_{s} - {u}_{d} )}^{2} \cdot {R}_{vir}^{2}}{( {R}_{i}^{2} - {R}_{i - 1}^{2} )} - ρ \cdot {u}_{d} \cdot ( {u}_{s} - {u}_{d} )$$

While the embodiments of the present disclosure have been described with respect to a limited number of embodiments, it will be appreciated that many variations, modifications and other applications of the embodiments may be made.

What is claimed is:

1. A non-invasive method for automatically and autonomously determining at least one of cardiac and hemodynamic parameters of a subject based on an ultrasound scan of a static scanning area over a chest of said subject, wherein said scan is performed without presenting an ultrasound image of said static scanning area to at least one of a user, practitioner, and caregiver, the method comprising:
a. scanning the static scanning area over the chest of the subject with an ultrasound probe comprising an array of a plurality of ultrasound transducers;
b. determining autonomously at least two vessel parameters for at least two vessels within said static scanning area; and
c. processing said at least two vessel parameters for at least one of said two vessels to determine said at least one of cardiac and hemodynamic parameters of said subject, said processing including:
d. obtaining a collection of points corresponding to a plurality of ultrasound signals detected and configured to be received from said array of ultrasound transducers, wherein said points correspond to locations about a surface of at least one of the vessels;
e. arranging said points on at least two axes;
f. confining all of said points within a quadrilateral about the two axes;
g. determining a center of said quadrilateral;
h. projecting a plurality of chords from said quadrilateral center to intersect with said points;
i. selecting a smallest chord from said plurality of projected chords intersecting with said points; wherein said smallest chord defines a diameter of said at least one of said two vessels and wherein said quadrilateral center defines a center of said at least one of said two vessels; and
j. converting each of said points by projecting them onto a circle defined by said vessel diameter and said vessel center.

2. The method of claim 1, wherein said scanning further comprises placing said ultrasound probe over the chest of the subject and maintaining said probe in a static position so as to continuously scan said static scanning area.

3. The method of claim 1, wherein said determining said at least two vessel parameters further comprises:
a. identifying two vessels within said static scanning area with said array of ultrasound transducers by automatic and autonomous means;
b. determining a center and a diameter of each of said two vessels based on said ultrasound scan;
c. activating a Doppler ultrasound signal within said scanning area and targeting said identified two vessels about their centers;
d. monitoring a plurality of Doppler flow parameters of a flowing fluid within each of said two vessels over a time period; and
e. processing said Doppler flow parameters and vessel parameters to identify cardiac and hemodynamic parameters.

4. The method of claim 3, wherein said time period is equivalent to at least four cardiac cycles or at least three consecutive cardiac cycles three.

5. The method of claim 3, wherein said two vessels are an aorta and a pulmonary artery.

6. The method of claim 3, wherein said two vessels are an ascending aorta and a pulmonary trunk.

7. The method of claim 3, wherein said processing said at least two vessel parameters further includes processing each of said two vessels individually to autonomously determine the center and diameter of each of said two vessels based on said ultrasound scan.

8. The method of claim 1, wherein said projecting a plurality of chords further comprises projecting a chord every 0.25 degrees to form 1440 chords about said quadrilateral center.

9. The method of claim 1, wherein said processing further comprises:
a. determining a velocity-time curve from a Doppler scan of said static scanning area;
b. identifying individual cardiac cycles within said velocity-time curve;
c. segmenting said velocity-time curve to correspond to a plurality of cardiac cycles to represent a plurality of cardiac cycle segments;
d. providing a plurality of sub-segments along each of said cardiac cycle segments;
e. determining parameters of said cardiac cycle segments based on said sub-segments; and
f. inferring a hemodynamic and cardiac parameter based on said cardiac cycle segment parameters.

10. The method of claim 9, wherein inferring said hemodynamic and cardiac parameter further includes:
a. determining an Area based on a vessel radius;
b. determining an area of the sub-segments based on the velocity-time curve;
c. determining a blood volume of the sub-segments;
d. determining a blood mass based on said blood volume of the sub-segments;
e. determining a blood flow acceleration of the sub-segments;
f. determining a blood flow force of the sub-segments; and
g. determining a pressure of the sub-segments of said velocity-time curve based on a ratio of said blood flow force and blood flow acceleration.

11. The method of claim 1, wherein determining autonomously at least two vessel parameters for at least two vessels within said static scanning area further comprises:
a. performing mask detection and filtering to identify elliptical object scanned within said static scanning area;
b. performing the Random Hough Transform (RHT) for an ellipse to identifying RHT associated parameters associated with objects identified within said scanning area;
c. performing further filtering and thresholding based on at least one RHT associated parameter;
d. activating a Doppler scan for at least one cardiac cycle to determine maximum blood flow velocity;
e. identifying said at least two vessels based on said blood flow velocity; and
f. scanning and monitoring said identified two vessels to determine at least two vessel parameters for at least three cardiac cycles.

12. The method of claim 11 wherein said RHT derived parameters are selected from the group consisting of major axis, minor axis, center, scan angle, and any combination thereof.

13. The method of claim 11 wherein said RHT derived parameters is further processed to determine the vessel objects boundaries selected from the group consisting of top, bottom, left and right.

14. The method of claim 11 wherein said identifying two vessels within said static scanning area comprises applying a threshold for vessel diameter and blood flow speed.

15. The method of claim 11 wherein said mask detection and filtering to identify elliptical object scanned within said static scanning area is selected from the group consisting of rectangular mask filtering, edge detection, boundary estimation, object shape threshold, size threshold, or any combination thereof.

16. The method of claim 11 wherein said determining autonomously at least two vessel parameters further comprises:
a. determining RHT derived parameters for said identified vessel comprising top boundary, bottom boundary, left boundary, right boundary and vessel center;
b. performing a border ultrasound scan at each vessel of said vessel boundary top boundary, bottom boundary, left boundary, right boundary utilizing a plurality of ultrasound scan lines for a period of time to monitor vessel location over time;
c. performing a Doppler scan of said RHT derived vessel center for a period of time to determine blood flow velocity over time;
d. tabulating said at least two vessel parameters including blood flow velocity and vessel center, and a time stamp for further processing; and
e. performing the RHT for said border ultrasound scan data to monitor vessel location coordinates over time.

17. The method of claim 16, wherein said plurality of ultrasound scan lines is preformed with 7 scan lines at each border centered about the RHT derived boarder.

18. The method of claim 16, wherein said time period is equivalent to at least three cardiac cycles.

19. The method of claim 16, wherein said border ultrasound scan time or said Doppler scan time is about 10 milliseconds.

20. The method of claim 1, wherein said processing provides for processing said at least two vessel parameters including vessel radius and blood flow velocity over time to determine at least one of cardiac or hemodynamic parameters of said subject, the method further comprising:
a. graphing vessel radius vs. time game including the systolic radius (Rsys) and diastolic radius (Rdia) points;
b. determining the slope between the Systolic radius (Rsys) and Diastolic radius (Rdia) points;
c. extrapolating the slope to identify the R_{P7} point wherein the slope intercepts with the time line axis;
d. extending a normal from Rsys point to intersect with the time line axis to determine the systolic time interval (tsys), and the pulse pressure time interval (tpp);
e. calculating pulse pressure (Ppulse) from the blood flow velocity vs. time graph;
f. evaluating a ratio Tpp/Tsys=Ppulse/Psys to calculate Psys; and
g. calculating Pdia by evaluation Pdia=Psys−Ppulse.

21. The method of claim 1, wherein said processing provides for processing said at least two vessel parameters including vessel radius and blood flow velocity, over time to determine cardiac or hemodynamic parameters of said subject, the method further comprising:
a. graphing vessel radius vs. time game;
b. providing a combination of Moens-Korteweg equation, Euler equation, Elasticity equation to define pressure as a function of vessel radius and blood flow velocity;
c. evaluating said pressure function with a plurality of measured data including vessel radius and blood flow velocity depicted in the vessel radius vs. time graph, to estimate a plurality of vessel parameters including Rvir, k, λ; and
d. calculating Ppulse, Pdia, Psys based on said estimated vessel parameters and measured data.

22. The method of claim 21 wherein said vessel radius and blood flow velocity data are provided every 10 milliseconds for at least three consecutive cardiac cycles.

23. The method of claim 21 wherein said plurality of vessel parameters including Rvir, k, λ are estimated by applying the least squares method.

24. A non invasive ultrasound system for automatic and autonomous determination of at least one of cardiac and hemodynamic parameters, the system configured to present an image of a scanned area to a user and wherein the scanned area is a static area over a chest of the user, the system comprising:
an ultrasound probe including a plurality of ultrasound transducers for scanning said static area over the chest;
a probe scan engine for controlling said plurality of ultrasound transducers and for processing data obtained from said ultrasound transducers to produce a set of vessel parameters; and
a processor configured to determine said at least one of cardiac and hemodynamic parameters from said vessel parameters by
a. scanning the static scanning area over the chest of the subject with an ultrasound probe comprising an array of a plurality of ultrasound transducers;
b. determining autonomously at least two vessel parameters for at least two vessels within said static scanning area; and
c. processing said at least two vessel parameters for at least one of said two vessels to determine said at least one of cardiac and hemodynamic parameters of said subject, said processing including:
d. obtaining a collection of points corresponding to a plurality of ultrasound signals detected and configured to be received from said array of ultrasound transducers, wherein said points correspond to locations about a surface of at least one of the vessels;
e. arranging said points on at least two axes;
f. confining all of said points within a quadrilateral about the two axes;
g. determining a center of said quadrilateral;
h. projecting a plurality of chords from said quadrilateral center to intersect with said points;
i. selecting a smallest chord from said plurality of projected chords intersecting with said points; wherein said smallest chord defines a diameter of said at least one of said two vessels and wherein said quadrilateral center defines a center of said at least one of said two vessels; and
j. converting each of said points by projecting them onto a circle defined by said vessel diameter and said vessel center.

25. The system of claim 24, wherein said ultrasound transducer array includes eight ultrasound transducers.

26. The system of claim 25, wherein said eight ultrasound transducers are arranged such that six outer transducers are arranged in a hexagonal formation having six vertices and two inner transducers are arranged internally to said hexagonal formation and spanning two chords defined between said six vertices.
